(19) United States
(12) Patent Application Publication
(10) Pub. No.: US 2023/0212788 A1
(43) Pub. Date: Jul. 6, 2023

(54) NEW METHOD FOR AUTOMATED ON-DEMAND BIOMOLECULAR ARRAY SYNTHESIS

(71) Applicant: TECHNISCHE UNIVERSITAT DRESDEN, Dresden (DE)

(72) Inventors: Yixin ZHANG, Dresden (DE); Weilin LIN, Dresden (DE)

(21) Appl. No.: 17/913,709

(22) PCT Filed: Mar. 24, 2021
(86) PCT No.: PCT/EP2021/057520

§ 371 Date: Sep. 22, 2022

(30) Foreign Application Priority Data
Mar. 26, 2020 (DE) 10 2020 108 373.4

(51) Int. Cl. C40B 50/18; B01J 19/00; C07K 1/04; G01N 33/543

(52) U.S. Cl. CPC C40B 50/18; B01J 19/0046; C07K 1/047; G01N 33/54393; B01J 2219/0061; B01J 2219/00603; B01J 2219/00619; B01J 2219/00626; B01J 2219/00637; B01J 2219/00725

(57) ABSTRACT

The invention provides an amphiphilic coating for the direct and rapid synthesis of an array of peptides and small molecular compounds on a planar surface of a solid support, comprising a hydrophilic chemical structure and a lipophilic group, wherein said peptides and small molecular compounds differ from spot to spot from each other in the chemical structure, characterized in that said amphiphilic coating possesses low wettability to polar aprotic solvents used in the array synthesis; said amphiphilic coating possessing low wettability is designed that it can be converted to a coating possessing high wettability by hydrolysis of the lipophilic group; and said amphiphilic coating comprises an amino group for the reaction with an electrophilic reagent. The invention further provides a solid support comprising said amphiphilic coating and a method for method for the direct and rapid synthesis of an array of peptides and small molecular compounds on a planar surface of a solid support, wherein said planar surface of a solid support comprises said amphiphilic coating. Said method includes the enhancing of the wettability of a glass surface to organic solvents to realize automated on-demand biomolecular array synthesis comprising both, peptides and small molecular compounds. The amphiphilic surface can be switched to a hydrophilic surface, resulting in high density arrays suitable for protein- and cell-based screening.

### FIELD OF THE INVENTION

The invention provides an amphiphilic coating for the direct and rapid synthesis of an array of peptides and small molecular compounds on a planar surface of a solid support, comprising a hydrophilic chemical structure and a lipophilic group, wherein said peptides and small molecular compounds differ from spot to spot from each other in the chemical structure, characterized in that said amphiphilic coating possesses low wettability to polar aprotic solvents used in the array synthesis; said amphiphilic coating possessing low wettability is designed that it can be converted to a coating possessing high wettability by hydrolysis of the lipophilic group; and said amphiphilic coating comprises an amino group for the reaction with an electrophilic reagent. The invention further provides a solid support comprising said amphiphilic coating and a method for method for the direct and rapid synthesis of an array of peptides and small molecular compounds on a planar surface of a solid support, wherein said planar surface of a solid support comprises said amphiphilic coating. Said method includes the reduced wettability of a glass surface to organic solvents to realize automated on-demand biomolecular array synthesis comprising both, peptides and small molecular compounds. The amphiphilic surface can be switched to a hydrophilic surface, resulting in high density arrays suitable for protein- and cell-based screening.

### BACKGROUND OF THE INVENTION

To detect protein-ligand or cell-ligand interactions on a planar surface can provide the most direct measurement to evaluate a library of chemical compounds, as the binding of cells or fluorescently labelled proteins can be visualized optically. Array of ligands can be constructed in high density (e.g. 100 - 10000 features/cm²), thus reducing assay volume and reagent consumption.[1] Many array technologies have been developed[2], making them increasingly attractive as complimentary methods to the one-well-one-assay approach. For example, in the field of DNA and RNA analysis, oligonucleotide array represents one of the most important application of array technology[3]. However, one-well-one-assay (96- and 384-well) still remains the most common method in drug screening[4]. Most studies of cell-material interaction are also performed in well formats. Only a few biomaterial screenings in array format have been reported. [5-7]. The major challenge for applying array technology in drug screening is the fact that to synthesize an addressable library is far more cumbersome than to distribute solutions into different wells. An in situ array synthesis technology, not only compatible with peptide and combinatorial chemistry, but also possessing desired surface property suitable for protein- and cell-based screening assays, is therefore needed. Such a method would provide a flexible and versatile chemical biology tool for drug discovery and cell-based assays.

For a certain design of combinatorial library as array, either of small molecules or oligopeptides, the theoretical library size (all possible combinations) is often far beyond the lab capability of synthesis. For example, a 10-mer peptide library has > 3 x 10¹¹ different possible sequences. To construct a simple combinatorial chemical library composed of two different pharmacophores (e.g. each with 1000 different building blocks) and a linker (e.g. 20 spacers of different lengths or rigidities), the library (20 millions) is remarkably smaller than the 10-mer peptide library, but still impossible for array synthesis. It is possible to synthesize libraries of such sizes, however, not in the array form: by using split-and-pool method. Libraries of DNA, RNA, peptide, and small organic molecule of billions of compounds can be generated as a mixture. The mixture will be subjected to certain selection mechanism to identify the active structures. Such selection technologies often involve sophisticated processes and indirect readouts, more complex than the direct visualization of interaction with an addressable array. For example, using the one-bead-one-compound [8] or DNA-encoded chemical library[9, 10] approaches, large chemical libraries can be synthesized, and used for selection experiments. Because the libraries are not in an addressable form, complicated decoding processes are necessary to reveal the selected compounds.

Although it is difficult to synthesize arrays as large libraries, one can explore the combinatorial chemical space by introducing cycles of library design and synthesis. The structure-activity relationship resulted from screening the first library are used for the design of second library. While each library is not necessarily big (e.g. 100 - 1000 compounds), the knowledge accumulated through the cycles will provide us with a comprehensive structure-activity relationship, thus allowing us to identify the optimal combination within a large chemical space (defined by the combinatorial chemistry and building blocks). This approach, sometimes referred as genetic algorithm in the field of medicinal chemistry and fragment-based drug discovery, requires high throughput on-demand library synthesis. However, because of the cumbersome in-solution medicinal chemistry and complications associated with array synthesis (as discussed later), genetic algorithm has not yet been realized as a powerful and routine technology. Another problem associated with in situ biomolecular array synthesis is that the materials compatible with chemical synthesis is often not compatible with the following assays. Therefore, the resulting arrays are often not directly applicable for protein- and cell-based screening.

There are two general approaches for synthesizing molecule arrays: immobilization of pre-synthesized compounds and in situ synthesis on a surface. The first approach, with which most oligonucleotide/peptide/protein/antibody arrays are prepared, is relatively simple. Since chemical compounds can be purified before spotting/immobilization, the resulting arrays would also be superior in quality. Moreover, when large number of arrays with identical structures are required, this approach is more economical than in situ synthesis. As there is only one major reaction step (spotting/immobilization), array preparation is relatively straightforward, does not involve many complications associated with in situ synthesis (as discussed later). However, as in situ synthesis does not need a pre-synthesized library, it offers the highest flexibility, especially for de novo discovery requiring multiple rounds of library design, synthesis and screening.

There are two major techniques for on-demand array synthesis, both having their advantages and disadvantages. The SPOT synthesis concept pioneered by Ronald Frank consists in the stepwise synthesis of peptides on cellulose membrane using standard Fmoc-based peptide chemistry, [11, 12] compatible with protected amino acids as well as other carboxylic acid building blocks. The high porosity of cellulose materials makes the substrate ideal for solid phase synthesis, able to absorb reactants in the matrix and easy to wash. However, also because of the high porosity, it is difficult to achieve small feature size and high array density (typically 3/cm²) on cellulose membrane. The large feature size and 3D matrix also cause high protein consumption in the screening experiments. For cellular assays, the strong light scattering by cellulose fibers interferes with fluorescence-based imaging and data analysis[5]. The second approach, by printing polymer particles containing pre-activated building blocks, can generate high-density peptide arrays (up to 10,000/cm²) [13-18]. However, the high instrumentation cost has limited its wide use. As compared to SPOT technology, it is less flexible, applicable for a small collection of pre-activated building blocks such as 20 natural amino acids. Moreover, as full automation has been realized for the second approach, it still remains difficult for peptide array synthesis with the polymer particle printing approach.

### SUMMARY OF THE INVENTION

The ability to synthesize libraries of biomolecules as high-density array with high yield, and to apply the array directly to biochemical and cell biological screening can provide a powerful tool to the fields of biomaterials, chemical biology and pharmaceutical sciences. There are two major challenges in in situ biomolecular array synthesis: 1) multi-step combinatorial synthesis on planar surface with small feature size and high yield; and 2) a surface compatible not only with the chemistry but also with the following protein- or cell-based screening assays.

The problem of the invention aiming to address was therefore to provide a surface compatible not only with the chemistry but also with the following protein- or cell-based screening assays. In particular, a surface enabling the synthesis of libraries of biomolecules as high-density array with high yield, which can directly applied to biochemical and cell biological screening assays, shall be provided.

This problem is solved by the invention by providing an amphiphilic coating of a solid support such as glass surface, on which small droplets of organic solvent can be deposited with relatively large contact angle and inhibited motion, permitting multiple rounds of combinatorial synthesis of small molecular compounds and peptides. The wettability of the surface of the solid support is reduced for organic solvents to realize automated on-demand biomolecular array synthesis comprising both, peptides and small molecular compounds. After completing the array synthesis, the amphiphilic surface can be switched to a hydrophilic surface, resulting in high density arrays suitable for protein- and cell-based screening.

### BRIEF DESCRIPTION OF THE DRAWINGS

FIG. 1 shows the relationship between droplet contact angle and droplet spreading area on a planar surface. Assuming a droplet possesses the shape of a perfect spherical cap, the ratio of droplet spreading area to the spreading area of a droplet in the shape of perfect semi sphere (θ = 90 °) is plotted versus the contact angle. When θ > 48 °, R < 2.

FIG. 2 shows the surface coating with chitosan (A) and serine (B).

FIG. 3 shows the general synthetic strategy for modifications of both —OH and —NH₂ groups to generate amphiphilic surface.

FIG. 4a shows the synthetic strategy for chitosan modifications: lipid on —OH group and Fmoc-linker on —NH₂ group. The fatty ester can be hydrolyzed in ammonia solution, while the Fmoc-linker is used in solid phase synthesis.

FIG. 4b shows the synthetic strategy for serine modifications: lipid on —OH group and Fmoc-linker on —NH₂ group. The fatty ester can be hydrolyzed in ammonia solution, while the Fmoc-linker is used in solid phase synthesis.

FIG. 4c shows the synthetic strategy for serine modifications: lipid on —OH group and Fmoc-linker on —NH₂ group. The tertiary ester linker can be hydrolyzed by TFA, while the Fmoc-linker is used in solid phase synthesis.

FIG. 5a shows the dynamic contact angle measurements of DMSO and Sulfolane/DMSO (6:4) mixture on surfaces with different lipophilic groups.

FIG. 5b shows the general strategy for introducing lipophilic chain to the coating through acid-catalyzed addition reaction to form ether bond and THP-C16 modification of coating and hydrolysis by TFA.

FIG. 6 shows a tilted glass slide with solvents (after 22 cycles of synthesis).

FIG. 7 shows the cleavage of desthiobiotin conjugated to the coating through ester bond by ammonia, as compared with a non-cleavable amide bond linkage.

FIG. 8 shows the spotting of polar aprotic solvent DMSO on surface with piezo inkjet printing.

FIG. 9 shows the spotting of polar aprotic solvent DMSO on surface with contact printing.

FIG. 10 shows the binding of streptavidin to biotin/desthiobiotin/iminobiotin synthesized on the glass surface.

FIG. 11 shows the binding of cyclophilin A to biotin or cyclosporin A derivative (CsA) synthesized on the glass surface.

FIG. 12 shows the results of coupling efficiency investigations.

FIG. 13 shows the binding of calcineurin to peptide PVIVIT or cyclosporin A derivative (CsA) synthesized on the glass surface.

FIG. 14 shows the epitope mapping of monoclonal anti-Flag antibody.

FIG. 15 shows a small molecular array for the discovery of TNF-α binders. A. 400-member small molecular array probed by fluorescently labelled TNF-α. B. Inhibition of TNF-α cytotoxicity by T1-T5. C. Concentration dependent inhibition of TNF-α cytotoxicity by T3 and T4.

FIG. 16 shows the adhesion of L929 cells to peptides synthesized as array on glass surface.

FIG. 17 shows different surface properties of modified glass surface.

FIG. 18 linker and amino protecting group optimization. Minimum of six repeat units of β -Ala as linker is necessary to avoid steric hinderance. Compared to Boc as amino protection group, Fmoc as amino protection group is unstable during lipid coupling process.

FIG. 18a Coupling of biotin to four repeat units of β-Ala.
Biotin-(β-Ala)₄: without lipid, intensity 37880 ± 4460.
Biotin-(β-Ala)₄/C16: Fmoc deprotection, then biotin coupling, then C16 acid coupling, intensity 29153 ± 1746
C16Biotin-(β-Ala)₄: C16 acid coupling, then Fmoc deprotection, then biotin coupling, intensity 18820 ± 734

FIG. 18b Coupling of biotin to six repeat units of β-Ala.
Biotin-(β-Ala)₆: without lipid, intensity 21206 ± 621
Biotin-(β-Ala)₆/C16: Boc deprotection, then biotin coupling then C16 acid coupling, intensity 21789 ± 1765.
C16/Biotin-(β-Ala)₆: C16 acid coupling, then Boc deprotection, then biotin coupling, intensity 22718 ± 1281.

### DETAILED DESCRIPTION OF THE INVENTION

### Amphiphilic Coating

In a first aspect, the invention provides an amphiphilic coating for the direct and rapid synthesis of an array of peptides and small molecular compounds on a planar surface of a solid support, comprising a hydrophilic chemical structure and a lipophilic group, wherein said peptides and small molecular compounds differ from spot to spot from each other in the chemical structure, characterized in that
said amphiphilic coating possesses low wettability to polar aprotic solvents used in the array synthesis;
said amphiphilic coating possessing low wettability is designed that it can be converted to a coating possessing high wettability by hydrolysis of the lipophilic group; and
said amphiphilic coating comprises an amino group for the reaction with an electrophilic reagent, wherein said electrophilic reagent is preferably comprised in a solution.

An “amphiphile” in the context of the present invention is a chemical compound possessing both hydrophilic and lipophilic properties or groups. Such a compound is called amphiphilic or amphipathic. Common amphiphilic substances are soaps, detergents and lipoproteins.

The “lipophilic group” is typically a large hydrocarbon moiety, such as a long chain of the form CH₃(CH₂)ₙ-, with n > 4.

The “hydrophilic group” falls into one of the following categories:
i) Charged groups:
   Anionic: Examples, with the lipophilic part of the molecule represented by an R, are: carboxylates: RCO₂⁻; sulfates: RSO₄⁻; sulfonates: RSO₃⁻; phosphates: The charged functionality in phospholipids;
   Cationic. Examples: ammonium groups: RNH₃⁺.
ii) Polar, uncharged groups. Examples are alcohols such as diacyl glycerol (DAG), and oligoethyleneglycols.

Often, amphiphilic species have several lipophilic parts, several hydrophilic parts, or several of both. Proteins and some block copolymers are such examples.

As a result of having both lipophilic and hydrophilic portions, some amphiphilic compounds may dissolve in water and to some extent in non-polar organic solvents.

There are several examples of molecules that present amphiphilic properties. Hydrocarbon based surfactants are an example group of amphiphilic compounds. Their polar region can be either ionic, or non-ionic. Some typical members of this group are sodium dodecyl sulfate (anionic), benzalkonium chloride (cationic), cocamidopropyl betaine (zwitterionic) and 1-octanol (long chain alcohol, non-ionic). Many biological compounds are amphiphilic, e.g. phospholipids, cholesterol, glycolipids, fatty acids, bile acids, saponins, local anaesthetics etc.

“Wetting” or “wettability” as used herein relates to the ability of a liquid to maintain contact with a solid surface, resulting from intermolecular interactions when the two are brought together. The degree of wetting (wettability) is determined by a force balance between adhesive and cohesive forces. Wetting deals with the three phases of materials: gas, liquid, and solid. Wetting is important in the bonding or adherence of two materials. Wetting and the surface forces that control wetting are also responsible for other related effects, including capillary effects. The wetting is also influenced by the type of coating on a surface.

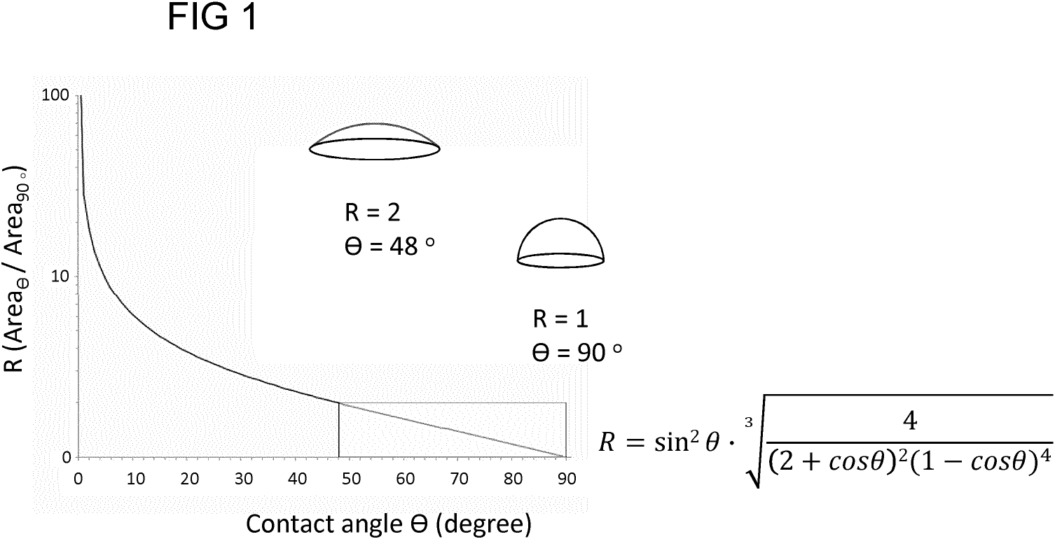

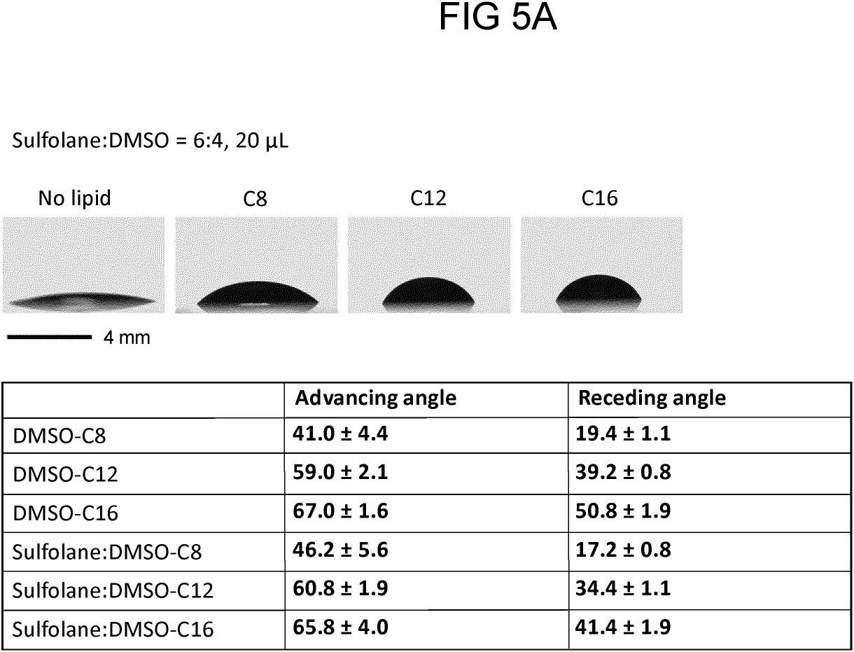

“Low wettability” in the context of the invention means that the amphiphilic coating possesses low wettability to polar aprotic solvents used in the array synthesis. Preferably, said amphiphilic coating is substantially not wettable with polar aprotic solvents used in the array synthesis. The difference in wettability can be measured using contact angle measurement (FIGS. 1 and 5). Low wettability leads to high value of contact angle. A contact angle (advancing angle) > 20 ° of a desired aprotic solvent to a surface is favorable for the array synthesis technology described in this invention.

“High wettability” in the context of the invention means that the amphiphilic coating possesses low wettability to not only to the organic solvents but also to water. Preferably, said amphiphilic coating is substantially wettable with organic solvents and water. In a most preferred embodiment, said amphiphilic coating is substantially wettable with organic solvents. In a further most preferred embodiment, said amphiphilic coating is substantially wettable with water. High wettability is characterized by a low value of contact angle between the aprotic solvent and the surface of the solid support. In this case, the contact angle is preferably < 20 ° of a desired aprotic solvent to a surface.

It is preferred in accordance with the present invention that the hydrophilic chemical structure for synthesizing the amphiphilic coating comprises both, at least one amino group and at least one hydroxyl group.

In a more preferred embodiment, hydrophilic chemical structure for synthesizing the amphiphilic coating is selected from an aminopolysaccharide and an amino acid.

The “aminopolysaccharide” is not particularly limited. In general, an aminopolysaccharide is any polysaccharide derived from an aminosugar. An amino sugar is a sugar molecule in which a hydroxyl group has been replaced with an amine group. More than 60 amino sugars are known, with one of the most abundant being N-Acetyl-d-glucosamine, which is the main component of chitin.

“Amino acids” include proteinogenic and non-proteinogenic amino acids as well as D- and L-amino acids. Proteinogenic amino acids are defined as natural protein-derived α-amino acids. Non-proteinogenic amino acids are defined as all other amino acids, which are not building blocks of common natural proteins. Also, [symbol] acids are included in the term “amino acids” according to the invention.

Examples of amino acids are aspartic acid (Asp), glutamic acid (Glu), arginine (Arg), lysine (Lys), histidine (His), glycine (Gly), serine (Ser) and cysteine (Cys), threonine (Thr), asparagine (Asn), glutamine (Gln), tyrosine (Tyr), alanine (Ala), proline (Pro), valine (Val), isoleucine (Ile), leucine (Leu), methionine (Met), phenylalanine (Phe), tryptophan (Trp), hydroxyproline (Hyp), beta-alanine (beta-Ala), 2-amino octanoic acid (Aoa), azetidine-(2)-carboxylic acid (Ace), pipecolic acid (Pip), 3-amino propionic, 4-amino butyric and so forth, alpha-aminoisobutyric acid (Aib), sarcosine (Sar), ornithine (Orn), citrulline (Cit), homoarginine (Har), t-butylalanine (t-butyl-Ala), t-butylglycine (t-butyl-Gly), N-methylisoleucine (N-MeIle), phenylglycine (Phg), cyclohexylalanine (Cha), norleucine (Nle), cysteic acid (Cya) and methionine sulfoxide (MSO), Acetyl-Lys, modified amino acids such as phosphoryl-serine (Ser(P)), benzyl-serine (Ser(Bzl)) and phosphoryl-tyrosine (Tyr(P)), 2-aminobutyric acid (Abu), aminoethylcysteine (AECys), carboxymethylcysteine (Cmc), dehydroalanine (Dha), dehydroamino-2-butyric acid (Dhb), carboxyglutaminic acid (Gla), homoserine (Hse), hydroxylysine (Hyl), cis-hydroxyproline (cisHyp), trans-hydroxyproline (transHyp), isovaline (Iva), pyroglutamic acid (Pyr), norvaline (Nva), 2-aminobenzoic acid (2-Abz), 3- aminobenzoic acid (3-Abz), 4- aminobenzoic acid (4-Abz), 4-(aminomethyl)benzoic acid (Amb), 4-(aminomethyl)cyclohexanecarboxylic acid (4-Amc), Penicillamine (Pen), 2-Amino-4-cyanobutyric acid (Cba), cycloalkane-carboxylic aicds.

Examples of [symbol] acids are e.g.: 5-Ara (aminoraleric acid), 6-Ahx (aminohexanoic acid), 8-Aoc (aminooctanoic aicd), 9-Anc (aminovanoic aicd), 10-Adc (aminodecanoic acid), 11-Aun (aminoundecanoic acid), 12-Ado (aminododecanoic acid).

Further amino acids are indanylglycine (Igl), indoline-2-carboxylic acid (Idc), octahydroindole-2-carboxylic acid (Oic), diaminopropionic acid (Dpr), diaminobutyric acid (Dbu), naphtylalanine (1-Nal), (2-Nal), 4-aminophenylalanin (Phe(4-NH2)), 4-benzoylphenylalanine (Bpa), diphenylalanine (Dip), 4-bromophenylalanine (Phe(4-Br)), 2-chlorophenylalanine (Phe(2-Cl)), 3-chlorophenylalanine (Phe(3-Cl)), 4-chlorophenylalanine (Phe(4-Cl)), 3,4-chlorophenylalanine (Phe (3,4-Cl2)), 3- fluorophenylalanine (Phe(3-F)), 4-fluorophenylalanine (Phe(4-F)), 3,4- fluorophenylalanine (Phe(3,4-F2)), pentafluorophenylalanine (Phe(F5)), 4-guanidinophenylalanine (Phe(4-guanidino)), homophenylalanine (hPhe), 3-jodophenylalanine (Phe(3-J)), 4 jodophenylalanine (Phe(4-J)), 4-methylphenylalanine (Phe(4-Me)), 4-nitrophenylalanine (Phe-4-NO2)), biphenylalanine (Bip), 4-phosphonomehtylphenylalanine (Pmp), cyclohexyglycine (Ghg), 3-pyridinylalanine (3-Pal), 4-pyridinylalanine (4-Pal), 3,4-dehydroproline (A-Pro), 4-ketoproline (Pro(4-keto)), thioproline (Thz), isonipecotic acid (Inp), 1,2,3,4,-tetrahydroisoquinolin-3-carboxylic acid (Tic), propargylglycine (Pra), 6-hydroxynorleucine (NU(6-OH)), homotyrosine (hTyr), 3-jodotyrosine (Tyr(3-J)), 3,5-dijodotyrosine (Tyr(3,5-J2)), d-methyl-tyrosine (Tyr(Me)), 3-NO2-tyrosine (Tyr(3-NO2)), phosphotyrosine (Tyr(PO3H2)), alkylglycine, 1-aminoindane-1-carboxy acid, 2-aminoindane-2-carboxy acid (Aic), 4-amino-methylpyrrol-2-carboxylic acid (Py), 4-amino-pyrrolidine-2-carboxylic acid (Abpc), 2-aminotetraline-2-carboxylic acid (Atc), diaminoacetic acid (Gly(NH2)), diaminobutyric acid (Dab), 1,3-dihydro-2H-isoinole-carboxylic acid (Disc), homocylcohexylalanin (hCha), homophenylalanin (hPhe oder Hof), trans-3-phenyl-azetidine-2-carboxylic acid, 4-phenyl-pyrrolidine-2-carboxylic acid, 5-phenylpyrrolidine-2-carboxylic acid, 3-pyridylalanine (3-Pya), 4-pyridylalanine (4-Pya), styrylalanine, tetrahydroisoquinoline-1-carboxylic acid (Tiq), 1,2,3,4-tetrahydronorharmane-3-carboxylic acid (Tpi), β-(2-thienryl)-alanine (Tha).

For peptide array synthesis, preferred according to invention are L-proteinogenic amino acids.

When the hydrophilic chemical structure of the amphiphilic coating is an aminopolysaccharide, said hydrophilic chemical structure is most preferably chitosan.

When the hydrophilic chemical structure of the amphiphilic coating is an amino acid, said hydrophilic chemical structure is most preferably serine.

In general, a “small molecule” is characterized by molecular weights of 1000 g/mole or less, preferably 800 g/mole or less, preferably of 500 g/mole or less, and even more preferably of 350 g/mole or less and even of 300 g/mole or less.

A “peptide” defines a biomolecule composed of amino acids linked by a peptide bond. The length of a peptide, i.e. the number of amino acids comprised in a peptide, may vary. Suitably, the peptides synthesized in the array of the invention comprise between 2 and 200 amino acids, preferably between 3 and 100, more preferably between 4 and 75, most preferably between 5 and 50 amino acids.

“Organic solvents” are classified as aliphatic hydrocarbons, cyclic hydrocarbons, aromatic hydrocarbons, halogenated hydrocarbons, ketones, amines, esters, alcohols, aldehydes, and ethers. Examples of organic solvents suitable for use in the present invention are selected from acetonitrile, chlorobenzene, chloroform, cyclohexane, 1,2-dichloroethene, dichloromethane, 1,2-dimethoxyethane, N,N-dimethylacetamide, N,N -dimethylformamide, 1,4-dioxane, 2-ethoxyethanol, ethylene glycol, formamide, hexane, methanol, 2-methoxyethanol, methylbutylketone, methylcyclohexane, N-methylpyrrolidone, nitromethane, pyridine, sulfolane, tetralin, toluene, 1,1,2-trichloroethylene and xylene.

A subgroup of organic solvents are aprotic solvents. “Aprotic solvents” are solvents that lack an acidic hydrogen. They are not hydrogen bond donors, but can accept hydrogen bonds. These solvents generally have intermediate dielectric constants and polarity. IUPAC describes such solvents as having both high dielectric constants and high dipole moments. Aprotic solvents can dissolve some salts.

Examples of aprotic solvents suitable for use in the present invention are acetonitrile, pyridine, ethyl acetate, DMF (dimethylformamide), HMPA (hexamethylphosphoramide), N-methyl-2-pyrrolidone (NMP), sulfolane and DMSO (dimethyl sulfoxide). Preferred aprotic solvents according to the invention are NMP, DMF, DMSO and sulfolane.

“Lipophilic group” is preferably lipid group, i.e. a fatty acid molecule or fluorinated fatty acid molecule coupled to hydroxyl groups of the hydrophilic chemical structure.

In a preferred embodiment of the invention, the modification of said amphiphilic coating with a lipophilic group is carried out through ester bond formation by coupling a fatty acid molecule or a fluorinated fatty acid molecule to the hydroxyl groups of the hydrophilic chemical structure, wherein at least one —OH (hydroxyl) group hydrophilic chemical structure is replaced by a carboxylic acid ester group, wherein the alkyl group is introduced by the fatty acid molecule. The resulting ester bonds are preferably labile to base-catalyzed hydrolysis.

In a further preferred embodiment of the invention, it is also possible to carry out the conjugation of lipophilic group through ether bond formation with the hydroxyl group of the hydrophilic chemical structure, to form an acid-labile ether linkage. In this embodiment, the hydroxyl groups of the hydrophilic chemical structure are preferably modified through an acid-catalyzed addition reaction to form the ether bond, while the resulting ether bond is labile to acid-catalyzed hydrolysis. A suitable compound for the formation of an ether linkage is dihydropyran, which forms with the hydroxyl group of the hydrophilic chemical structure an acid-labile tetrahydropyranyl ether.

“Fatty acids” are important components of lipids (fat-soluble components of living cells) in plants, animals, and microorganisms. Generally, a fatty acid consists of a straight chain of an even number of carbon atoms, with hydrogen atoms along the length of the chain (i.e. an alkyl chain) and at one end of the chain and a carboxyl group (—COOH) at the other end. It is that carboxyl group that makes it an acid (carboxylic acid). If the carbon-to-carbon bonds are all single, the acid is saturated; if any of the bonds is double or triple, the acid is unsaturated and is more reactive. A few fatty acids have branched chains; others contain ring structures (e.g., prostaglandins).

Accordingly, the fatty acid used in the amphiphilic coating of the present invention may be selected from saturated, unsaturated, straight chain, branched or cyclic fatty acids. Preferred are straight chain saturated fatty acids.

In a further preferred embodiment, the amphiphilic coating of the invention comprises a lipophilic group, which comprises an alkyl chain of 4-20 carbon atoms, preferably of 6 to 18 carbon atoms, more preferably of 8 to 16 carbon atoms. Most preferably, the lipophilic group comprises 8, 12 or 16 carbon atoms.

In a further preferred embodiment, the amphiphilic coating of the invention comprises on the surface of the solid support a linker between the amphiphilic coating and the amino group, which is used for subsequent array synthesis. More preferably, said linker is a poly-amino acid linker. Most preferably, said poly-amino acid linker has the formula (aa)ₙ, wherein aa is an amino acid or a protected amino acid and n is an integer between 3 to 10. The amino acid aa is preferably selected from the group consisting of glycine, beta-alanine, lysine, serine, threonine, aspartic acid and glutamic acid, wherein said amino acid can optionally be protected. The linker can consist of n monomers of the same amino acid or protected amino acid listed above or of a combination of amino acids and protected amino acid selected from the above mention groups. In a preferred embodiment, the linker consists of n monomers of the same amino acid or protected amino acid.

The length of the linker and the stability of amino protecting group in the lipid coupling process have an influence on the steric hindrance of the lipophilic groups and affect the protein-ligand interaction. In other words, if the linker is too short, steric hindrance caused by the lipophilic group cannot be avoided. Accordingly, in a preferred embodiment, n is an integer selected from 4, 5, 6, 7, 8, 9 and 10, more preferably an integer selected from 4, 5, 6, 7 and 8, most preferably an integer selected from 5, 6 and 7. In further most preferred embodiment, n is 6.

Suitable protection groups of the side chains in the linker are tBu (tert-Butyl) and Boc (tert-Butyloxycarbonyl). Most preferably, the protection group of lysine side chain is Boc. The most preferred protection group of serine, threonine, aspartic acid, glutamic side chains is tBu. Accordingly, the amino acid aa in linker is preferably selected from Boc-protected lysine, glycine, beta-alanine, tBu-protected serine, tBu-protected threonine, tBu-protected aspartic acid and tBu-protected glutamic acid.

In a most preferred embodiment, the linker consists of β-Ala and n is 6.

When the modification of said amphiphilic coating with a lipophilic group is carried out through ester bond formation, said amphiphilic coating typically possesses a remarkably reduced wettability to various polar aprotic organic solvents, including DMSO, DMSO/sulfolane mixture. Through the ester bonding of the lipophilic group, said amphiphilic coating is designed in a way that it can be converted to a coating possessing high wettability by hydrolysis of the lipophilic group with a base.

Suitable bases for changing the wettability of the amphiphilic coating of the invention, when the lipophilic group is coupled to the hydrophilic structure through an ester bond, are selected from hydroxides of alkali or alkaline earth metals; or a substances that produce hydroxide ions in aqueous solutions (so-called Arrhenius bases). Further suitable bases do not contain a hydroxide ion but nevertheless react with water, resulting in an increase in the concentration of the hydroxide ion. An example thereof is ammonia. Hydroxides of alkali or alkaline earth metals as well as Arrhenius bases are well known to the person skilled in the art.

Suitable bases are selected from lithium hydroxide, sodium hydroxide, potassium hydroxide, rubidium hydroxide, cesium hydroxide, calcium hydroxide, strontium hydroxide, barium hydroxide, tetramethylammonium hydroxide, guanidine, butyl lithium, lithium diisopropylamide, lithium diethylamide, sodium amide, sodium hydride, lithium bis(trimethylsilyl)amide and ammonium.

In a preferred embodiment, the amphiphilic coating can be switched to a hydrophilic coting by incubation of the coating in a solution of a base as mentioned before, more preferably an ammonia solution, to hydrolyze the ester bond.

When said conjugation of lipophilic group in said amphiphilic coating is carried out through ether bond formation, said amphiphilic coating typically possesses a remarkably reduced wettability to various polar aprotic organic solvents, including DMSO, DMSO/sulfolane mixture. Through the ether bonding of the lipophilic group, said amphiphilic coating is designed in a way that it can be converted to a coating possessing high wettability by hydrolysis of the lipophilic group with an acid.

An “acid” according to the invention is a molecule or ion capable of donating a proton (hydrogen ion H+) (a Brønsted-Lowry acid), or, alternatively, capable of forming a covalent bond with an electron pair (a Lewis acid). The acid can also be an Arrhenius acid, which is a substance that, when added to water, increases the concentration of H⁺ ions in the water. Brønsted-Lowry acids, Lewis acids and Arrhenius acid are well known to the person skilled in the art. Common acids are mineral acids, sulfonic acids, carbocyclic acids, halogenated carbocyclic acids, and vinylogous carbocyclic acids (e.g. ascorbic acid).

Suitable examples of mineral acids include hydrofluoric acid, hydrochloric acid, hydrobromic acid, hydroiodic acid, hypochlorous acid, chlorous acid, chloric acid, perchloric acid, and corresponding analogs for bromine and iodine, hypofluorous acid, sulfuric acid, fluorosulfuric acid, nitric acid, phosphoric acid, fluoroantimonic acid, fluoroboric acid, hexafluorophosphoric acid, chromic acid and boric acid.

Suitable examples of sulfonic acids include methanesulfonic acid, ethanesulfonic acid, benzenesulfonic acid, p-toluenesulfonic acid, trifluoromethanesulfonic acid, and polystyrene sulfonic acid.

Suitable examples of carboxylic acids include acetic acid, citric acid, formic acid, gluconic acid, lactic acid, oxalic acid, and tartaric acid.

Suitable examples of halogenated carboxylic acids include fluoroacetic acid, trifluoroacetic acid, chloroacetic acid, dichloroacetic acid, and trichloroacetic acid.

In a preferred embodiment, the amphiphilic coating comprising an ether bonding of the lipophilic group, can be switched to a hydrophilic coting by incubation of the coating in an acid solution as mentioned before, more preferably in trifluoroacetic acid (TFA).

### Solid Support

In a further aspect, the invention provides a solid support comprising a planar surface coated with an amphiphilic coating as described hereinbefore.

In a preferred embodiment, said solid support is made from a non-porous material.

In a more preferred embodiment, said non-porous solid support is glass.

Glass has several advantages: Though array synthesis on cellulose membrane offers the highest flexibility, it is difficult to produce small feature size, preventing the generation of high-density arrays. Moreover, because of the high protein consumption and light scattering caused by cellulose matrices, glass represents a surface superior to cellulose membrane for high throughput screening.

The glass as solid support comprising the amphiphilic coating of the invention is particularly advantageous. It is an ideal surface for in situ on-demand array synthesis, which fulfills the following requirements: 1) For generating small droplets to create high density array, the surface has relatively low wettability to aprotic polar organic solvents. 2) The surface has some binding energy to the droplets, to prevent droplet movement such as vapor-mediated droplet motion. 3) The final surface is, after switching the wettability, hydrophilic and compatible with various biochemical and cellular assays.

The amphiphilic coating of the glass surface allows the deposition of small droplets of an organic solvent, preferably a polar aprotic solvent, which can be deposited with a relatively large contact angle and inhibited motion, permitting multiple rounds of combinatorial synthesis of small molecular compounds and peptides.

Organic solvents, such as DMF, cannot form droplets on normal glass surface or on amino-functionalized glass surface, as their high wettability to the substrate leads to small contact angle (θ ≤ 10°), causing spread of the liquid over a large area. While a small volume of solution in the shape of hemisphere (θ = 90°) can be considered as an ideal droplet for directing chemical reaction to defined position on a surface (with area A₀), the plot in FIG. 1A depicts the relationship between contact angle and spot size (with area A) for a droplet of certain volume, when θ ≤ 90°. (When θ > 90°, the initial contacting area could be smaller. However, upon surface modification, the surface energy and contact angle could also change. Thus, the final spot area cannot be predicted. When (θ ≤ 90°, the final spot area will be ≤ A₀.) When θ decreases, the area increases exponentially. A surface with relatively large contact angle for polar solvents such as DMF and sulfolane is favorable. For example, when θ > 48°, the resulting spot will not be more than two times larger than that from a hemisphere droplet. On the other hand, the droplet should not possess a very large contact angle, especially the receding angle. Diminishing the wettability to a surface, for example with a Teflon coating, would cause the solvent droplets moving on the surface with little resistance upon small disturbance in their environment, which is not desired for directed array synthesis.

The amphiphilic coating on the glass surface is not only compatible with high density spotting technology using different polar aprotic organic solvents, but also applicable for solid phase chemical syntheses, e.g. of various small molecular ligands and peptides. The amount of amino groups necessary for small molecular ligands and/or peptide synthesis can be tuned in the coating during the formation of the coating. Accordingly, the invention provides in further preferred embodiment a solid support, wherein the solid support has a surface-specific loading with amino groups in the range of 1 pmol to 100 nmol per cm², preferably between 25 pmol and 50 nmol per cm², more preferably between 50 pmol and 10 nmol per cm², most preferably between 70 pmol and 2 nmol per cm².

### Synthesis of the Amphiphilic Coating

In a further aspect, the invention relates to a method for synthesizing the amphiphilic coating of the invention. The amphiphilic coating is synthesized on the solid support. Suitably, a hydrogel comprising an aminopolysaccharide is conjugated to the solid support, such as a glass support as follows: By treating an amino-silanized glass slide with an anhydride, the amino-functionalized surface is converted to carboxylic acid-functionalized surface. After activating the carboxylic groups with 1-ethyl-3-(3-dimethylaminopropyl)carbodiimid (EDC)/N-hydroxysuccinimide (NHS), said aminopolysaccharide is added to form a coating by crosslinking the carboxylic groups on the surface of the solid support with the amino groups of the aminopolysaccharide. The remaining amino groups are then coupled with an amino acid, which is preferably protected, such as glycine, beta-alanine, Boc protected lysine, tBu protected serine, tBu protected threonine, tBu protected aspartic acid and tBu protected glutamic acid, using a coupling reagent, such as hexafluorophosphate azabenzotriazole tetramethyl uronium (HATU)/(N-methylmorpholine (NMM), while the hydroxyl groups are left for introducing lipophilic modifications into the amphiphilic coating, in order to tune the wettability of surface to organic solvents. In a preferred embodiment, the amphiphilic coating comprises on the surface of the solid support a linker between the amphiphilic coating and the (optionally protected) amino acid in order to avoid steric hindrances in the subsequent array synthesis. A surface with both hydroxy and amino groups can also be generated through coupling a protected amino acid, such as Fmoc-Ser-OH to an amino-modified surface of the solid support, followed by deprotection reaction, such as Fmoc deprotection. There are formed either ester bonds or ether bonds by adding the lipophilic groups, wherein the resulting ester bonds are labile to base-catalyzed hydrolysis and the resulting ether bonds are labile to acid-catalyzed hydrolysis.

### Array Synthesis

In a further aspect, the invention relates to a method for the direct and rapid synthesis of an array of peptides and/or small molecular compounds on a planar surface of a solid support, wherein said planar surface of a solid support comprises an amphiphilic coating according to the invention, or wherein said rapid synthesis is one of as described herein, characterized in that said method comprises the steps:
a) covalently bonding to the amino groups of the amphiphilic coating in predetermined discrete spotting zones, the starting building blocks of the peptides and small molecular compounds to be synthesized, by spotting droplets of a solution comprising chemical reagents reactive to the amino groups onto the predetermined discrete spotting zones, and
b) synthesizing chemical compounds by reacting the first building block with further reactants, in a predetermined sequence and at the predetermined discrete spotting zone, by spotting droplets of a solution comprising chemical reagents reactive to the first building block;
c) obtaining a unitary, single solid support comprising an array of different, combinatorically synthesized, bound peptides and/or small molecular compounds,
wherein the chemical structure of said peptides and/or small molecular compounds in said array is different from spot to spot.

“Unitary” in the context of the invention means that planar surface is of high quality without defective areas and that the spotting zones are equally distributed on the planar surface.

In a preferred embodiment, said amphiphilic coating possesses low wettability to polar aprotic solvents used in the array synthesis. Even preferably, the contact angle of the polar aprotic solvent to the surface is > 20°. This ensures formation of small discrete droplets of the aprotic solvents in high density on the solid support. In a most preferred embodiment, a droplet, that covers a predetermined discrete spotting zone, has a diameter in the range of 1 µm to 2 mm, preferably 10 µm to 1 mm, more preferably 50 µm to 750 µm, most preferably 100 µm to 500 µm.

The aprotic solvent used in the array synthesis is preferably selected from NMP, DMF, DMSO and sulfolane.

Said unitary, nonporous solid support having a planar surface, preferably comprises a plurality of separate, designated spotting zones.

When a peptide array shall be synthesized, each spotting zone suitably comprises chemically reactive groups to which the C-terminus of a peptide under synthesis can be covalently bonded. The synthesis of the peptide array comprises covalently bonding to the reactive groups in each freely selected discrete spotting zone, a starting amino acid residue of the sequence of each peptide to be synthesized by adding to each freely selected discrete spotting zone a solution of an activated N-protected derivative of the starting amino acid residue, and synthesizing the different peptides by coupling additional amino acid residues to the starting amino acid residue, in a predetermined sequence, by adding to each predetermined discrete spotting zone solutions of activated N-protected derivatives of the amino acids according to said predetermined sequence, whereby there is obtained a unitary, single solid support comprising a plurality of different, bound peptides.

The linking of amino acids to peptides takes place stepwise, beginning with the C-terminal, and, where appropriate, in parallel for many peptides by the iteration of the same three reaction steps on each occasion. The linking is performed as follows:
1) Peptide bond formation: A fresh mixture (40 pL to 200 nl) of amino acid/HATU/NMM (molar ratio 1:1:3) solution is deposited to a predetermined position on the planar solid support. As a result, a reaction area (patch) is formed which is defined by the volume applied. In this solution, the amino acids comprise a protective group at the N-terminus. If many different peptides are to be synthesized in parallel on a correspondingly large support surface, the sites for application are located at distances which ensure that these reaction areas cannot intersect. The reaction time is 30 to 60 minutes, for example. After this, the support is washed three times with following solvents (10% acetic acid, 45% DMF, 45% ethanol, then 50% DMF and 50% ethanol, then pure ethanol). The solvents on the support are dried under vacuum. The coupling process is repeated one time.
2) Capping of unreacted amino groups. The support is treated with mixture of equal volume of Cap A (10% acetic anhydride in DMF) and Cap B (4% DIPEA in Ethanol) for 30 mins. After this, the support is washed three times with following solvents (50% DMF and 50% ethanol, then ethanol). The solvents on the support are dried by vacuum.
3) Elimination of the N-terminal protective group: for this purpose, the support is treated with sufficient cleavage or deprotection solution. When the preferred Fmoc/tBu method is implemented, the Fmoc protective group is eliminated with 20 percent piperidine in DMF/ethanol (volume 1:1) for 30 minutes. After this, the support is washed three times with following solvents (50% DMF and 50% ethanol, then pure ethanol). The solvents on the support are dried by vacuum.

After the completion of peptide sequences, the protective groups on the amino acid side chains can be eliminated by a suitable acid treatment (depending on the chosen synthesis method). For this purpose, the support is treated with cleavage solution. According to a special embodiment using the Fmoc/tBu method (see the following list of amino acid derivatives employed), treatment is carried out, for example, for 120 minutes with 88 % trifluoroacetic acid (TFA), 5 % water, 5 % Dithiothreitol and 2 % triisopropylsilane Thereafter, the support is washed several times with dichloromethane, then DMF, and then ethanol, and air-dried.

The array synthesis can be realized not only through peptide bond formation, but also through other chemical reactions in an aprotic solvent. Other chemical reactions include Baylis-Hillman reaction, Diels-Alder reaction, 1,3-Dipolar cycloaddition, Henry reaction, olefin metathesis, multiple component reaction including Ugi reaction and Passerini reaction, Nitroaldol reaction, Nozaki-Hiyama coupling, Paal-Knorr Pyrrole synthesis, Prins reaction, Sonogashira coupling, Staudinger synthesis, Stetter reaction [34], as well as various nucleophilic additions and nucleophilic substitutions, which all are conventional reactions and which are known to the person skilled in the art.

As described above, it may be necessary to perform blocking and deprotection steps during array synthesis. Accordingly, in a preferred embodiment of the array synthesis method, blocking and deprotection steps are carried out during synthesis. Most preferably, these blocking and deprotection steps are carried out on the entire surface of the solid support comprising the amphiphilic coating of the invention.

In a further preferred embodiment, the array synthesis method according to the invention further comprising as step d) the converting of the amphiphilic coating possessing low wettability into to a coating possessing high wettability by hydrolysis of the lipophilic group with a base or acid as described above. This advantageously allows the application of resulting arrays directly in screening.

### Uses

In a further aspect, the invention relates to the use of the unitary, single solid support comprising an array of different, combinatorically synthesized, bound peptides and small molecular compounds as produced with the method described above for the detection and/or identification of protein binding compounds, biomaterials and enzyme substrates and in cell adhesion assays.

In addition to achieving small droplets of organic solvent on glass with desired contact angle, compatibility with both chemical syntheses and biochemical assays represents another advantageous feature for developing on-demand array synthesis, as it would allow the application of resulting arrays directly in screening. Many resins used in solid phase synthesis show a high swelling degree for many organic solvents. However, the high compatibility with organic solvents also makes them intrinsically different from the hydrated network of tissues, thus unsuitable for most protein- and cell-based experiments. Therefore, the glass surface coating of the present invention makes the substrate compatible with classical solid phase combinatorial chemistry, including Fmoc-based peptide synthesis. By introducing a lipid chain or its fluorinated derivative into the coating, the surface wettability to organic solvents can be modulated and in situ array synthesis with small feature size (~ 50 µm) can be realized. Moreover, the amphiphilic coating can be switched to a hydrophilic matrix after the synthesis, to make the array suitable for protein binding and cell adhesion assays.

The invention is further described in more detail in following working examples.

### Example 1 - Synthesis of an Amphiphilic Coating on a Glass Surface

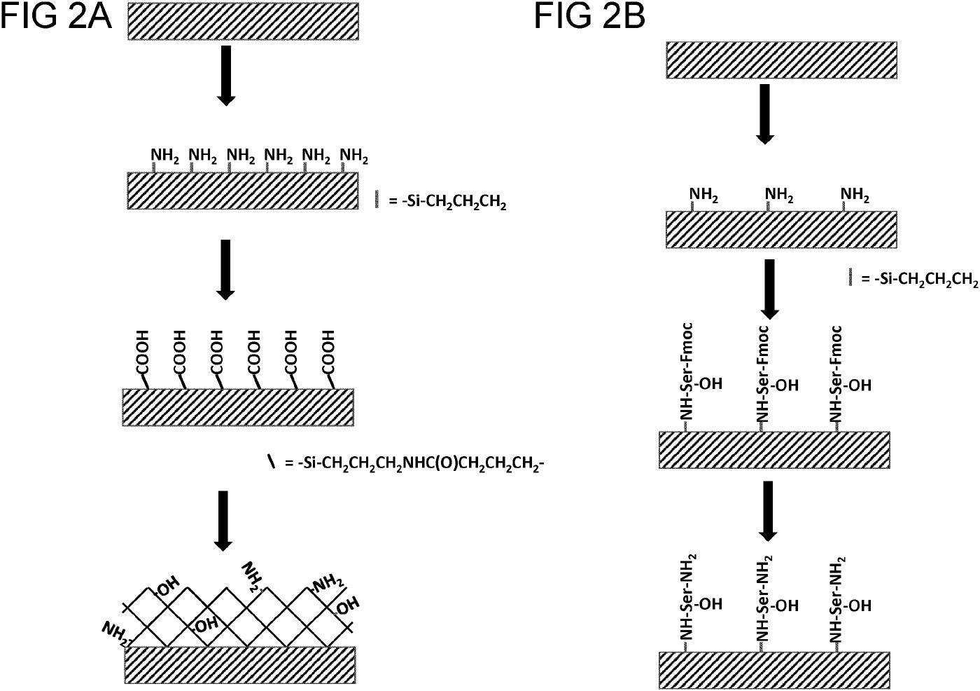

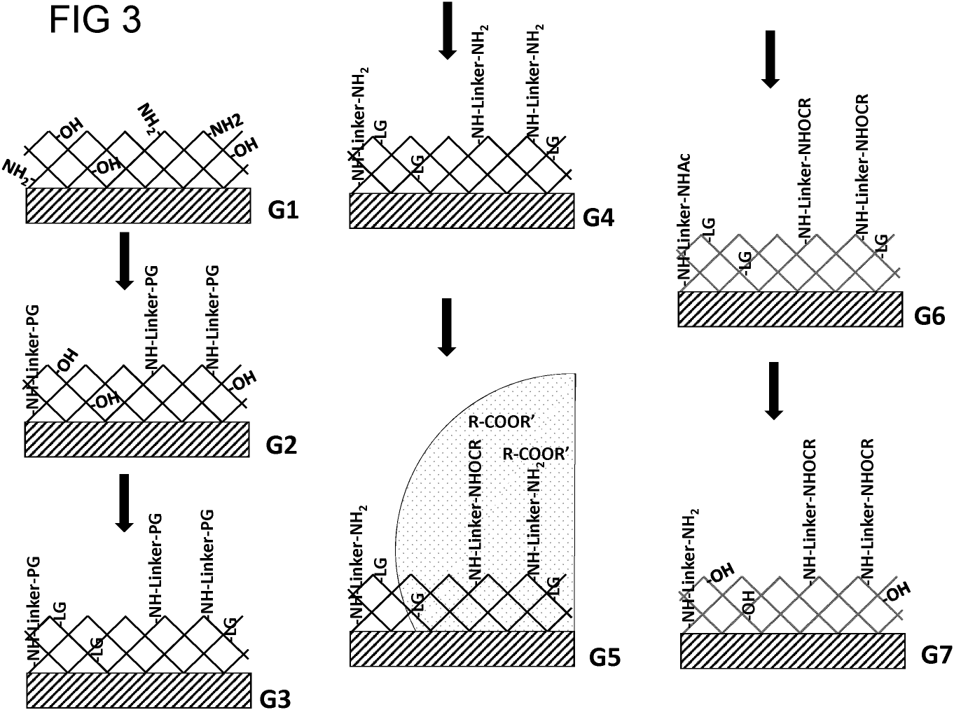

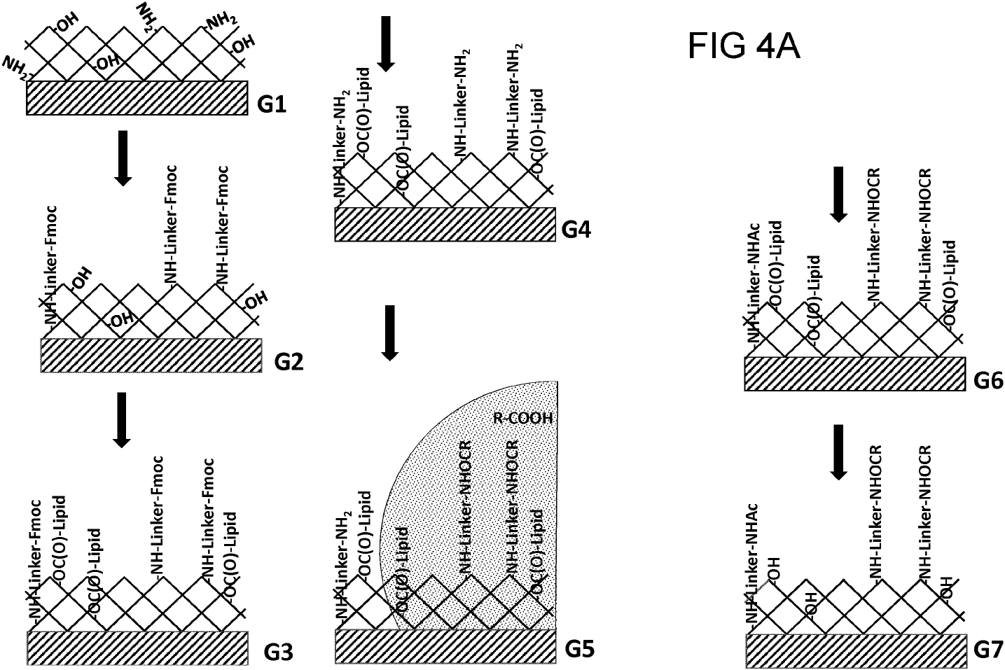

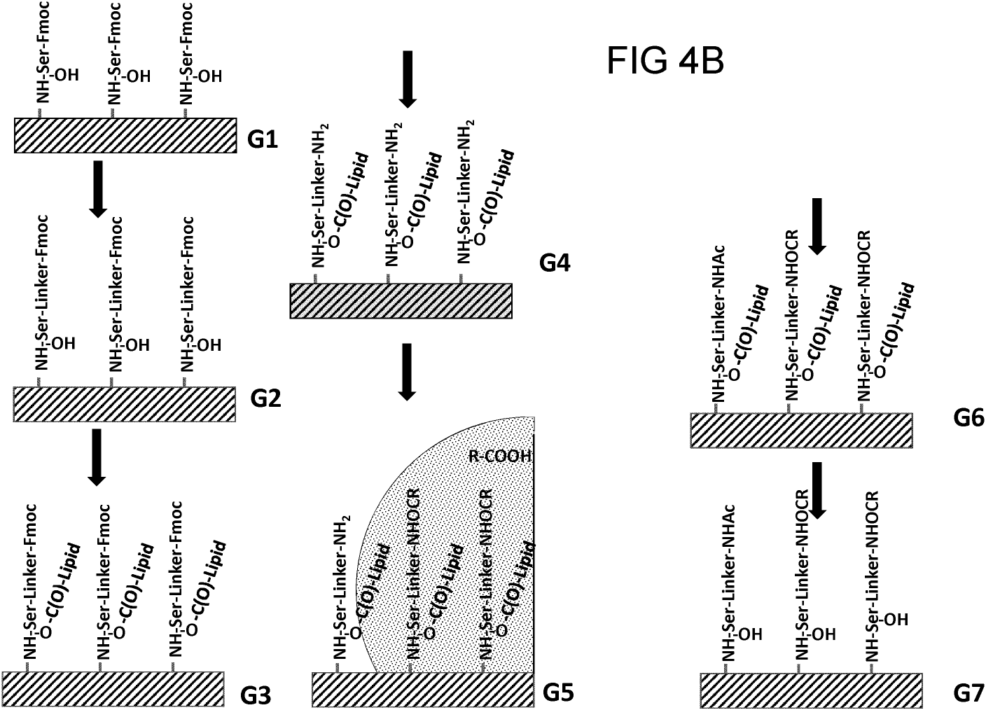

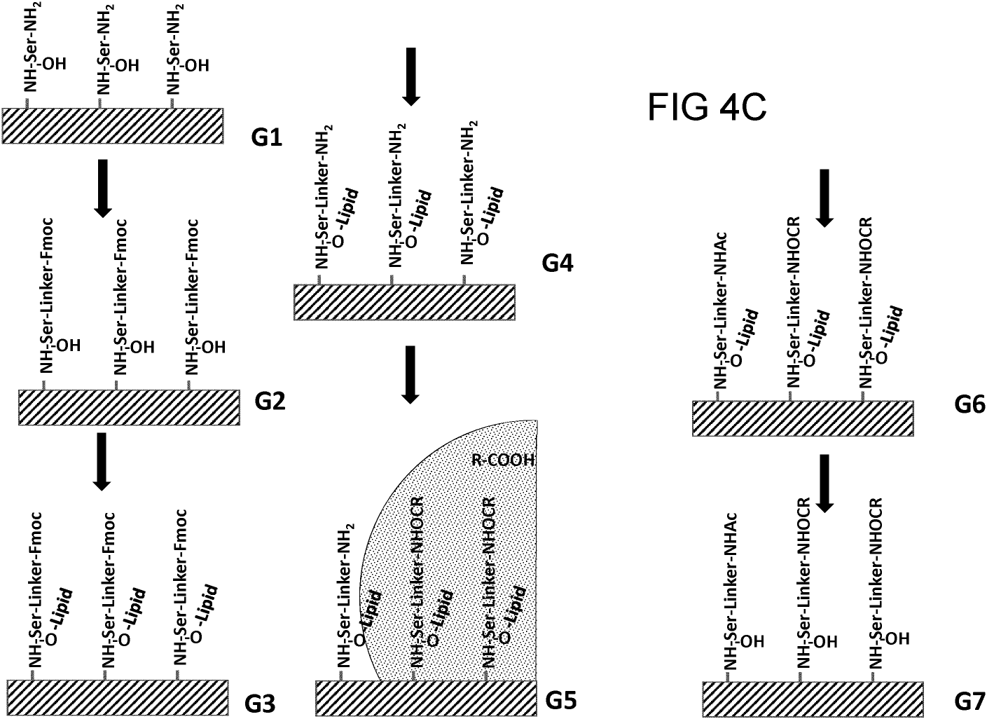

As depicted in FIG. 2, chitosan hydrogel coating was synthesized on glass surface. By treating amino-silanized glass slide with succinic anhydride, the amino-functionalized surface was converted to carboxylic acid-functionalized surface. After activating the carboxylic groups with EDC/NHS, chitosan was added to form a hydrogel coating by crosslinking the carboxylic groups on glass surface with the amino groups of chitosan. The remaining amino groups were then coupled with Fmoc-Gly using HATU/NMM as coupling reagent (G2), while the hydroxyl groups were left for introducing lipophilic modifications (G3), in order to tune the wettability of surface to organic solvents (FIGS. 3 and 4a). Alternatively, a surface with both hydroxy and amino groups can be generated through coupling Fmoc-Ser-OH to amino-modified glass surface, followed by Fmoc deprotection (FIG. 4b). Three fatty acids with 8, 12, and 16 carbons were chosen to modify the hydroxyl groups, using DIC/DMAP as coupling reagent, while the resulting ester bonds are labile to base-catalyzed hydrolysis. Alternative, the hydroxy group can be modified through acid-catalyzed addition reaction to form ether bond, while the resulting ether bond is labile to acid-catalyzed hydrolysis (FIG. 4c).

### Example 2: Amphiphilic Surface With Decreased Wettability to Organic Solvent

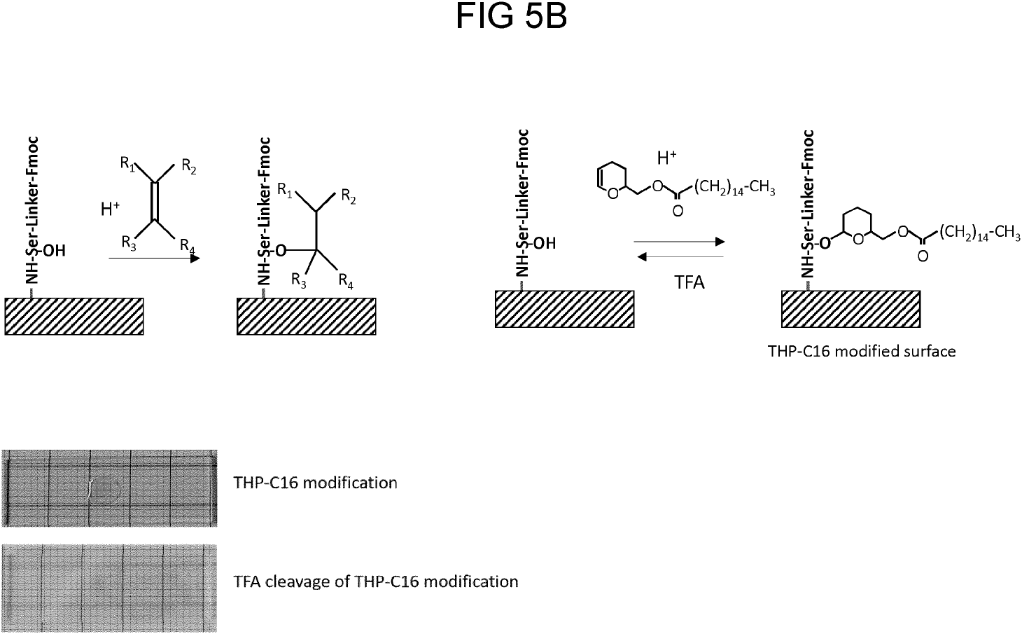

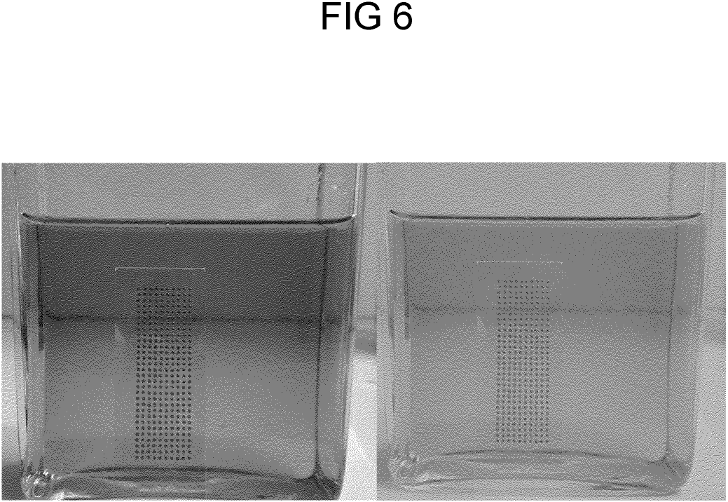

After the modification, the surfaces (G4 in FIG. 4) have shown remarkably reduced wettability to various polar aprotic organic solvents, including DMSO and DMSO/sulfolane mixture (FIGS. 5a and 5b). As expected, the C16 chain has shown the most remarkable effect on the wettability, showing advancing angles of 67° and 65.8° for DMSO and DMSO/sulfolane, respectively. In contrast, without the lipid chain modification, the contact angle is << 10° and cannot be correctly measured. The high boiling points of DMSO and sulfolane make them particularly interesting for array synthesis because of the slow evaporation of droplets after their deposition on the surfaces. Moreover, although the solvent droplets possess low wettability on the surface, they can bind strongly to the substrate, as reflected by their relatively small receding angles (FIG. 5a). Consequently, the droplets do not move even when the slide was tilted to 90° (FIG. 6). Multi-droplet interactions can cause droplet motion[19], while controlled droplet movement is important in microfluidic liquid handling, on self-cleaning surfaces and in heat transfer. Undesired droplet movement is one of the major obstacles for array synthesis using standard solvents and reagents for solid phase synthesis. The use of polymer particle as reaction medium represents an indirect solution to avoid droplet motion. With the C16 modified amphiphilic G4 surface, organic solvents can be deposited (e.g. by contact printing or piezo inkjet printing) with relatively large contact angle as well as completely inhibited droplet motion.

### Example 3: Switching Surface Wettability

The amphiphilic surfaces (G4-G6) can be switched to hydrophilic surface after incubation of the slide in ammonia solution, to hydrolyze the ester bond (FIG. 5a). Alternatively, the acid-labile ether bond can be hydrolyzed by TFA (FIG. 5b). DMSO, sulfolane, as well as water exhibit very small contact angle on the surface (G7 in FIG. 4), reflecting the high wettability of the solvents after the hydrolysis. Similar to the surface without lipid chain modification, the contact angle is << 10° and cannot be correctly measured.

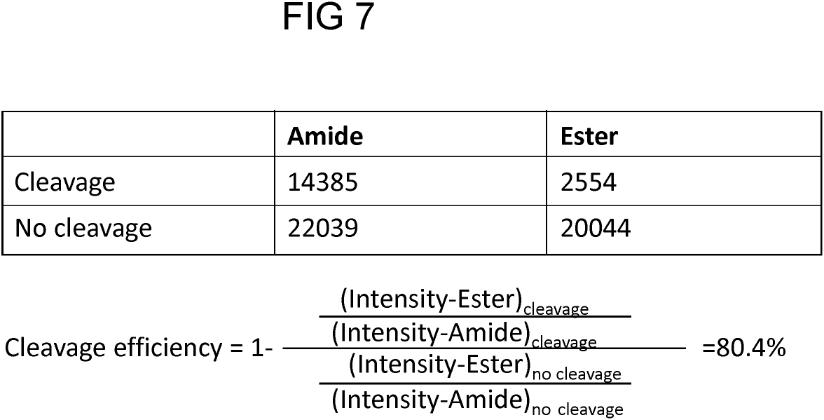

To measure the efficacy of hydrolysis/saponification reaction, streptavidin binder desthiobiotin was coupled to the hydroxy group using DIC/DMAP as coupling reagent (G3 in FIG. 3, where FG is desthiobiotin). Cy5-labelled streptavidin was used to monitor the formation and hydrolysis of ester bond. As a positive control, desthiobiotin was coupled to the amino group (G2 in FIG. 3, where PG is desthiobiotin). After treating the glass chip with ammonia solution, the G3 surface has shown an 80% decrease of signal intensity (FIG. 7), as compared to the G2 surface. Therefore, saponification with ammonia solution can efficiently hydrolyze the ester bond connected to chitosan matrix, while leaving the amide bond intact.

### Example 4: Droplet Deposition and Ligand Conjugation Through Printing

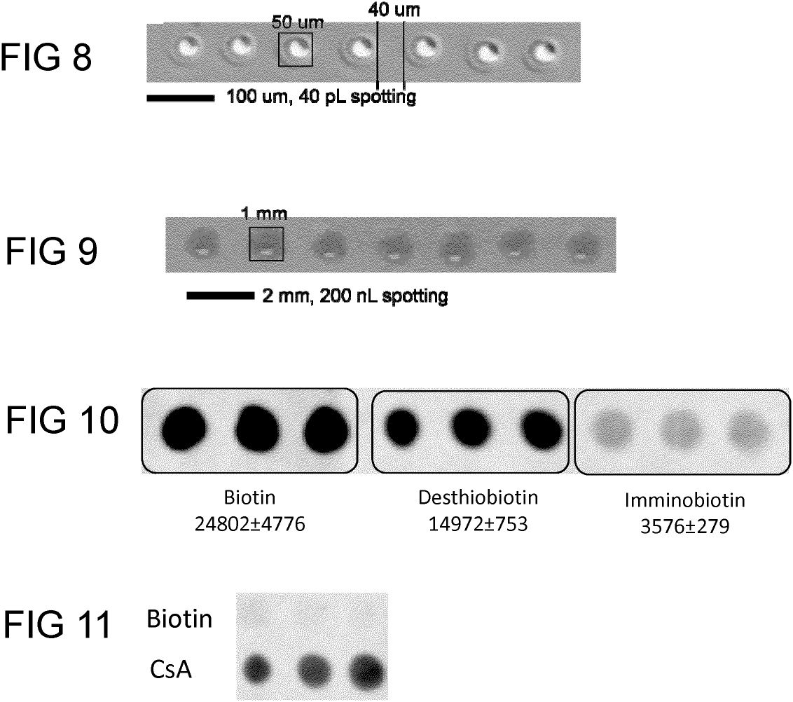

The substrate G4 (FIG. 4) can be used as solid support for solid phase combinatorial chemical syntheses. A carbolic acid (e.g. a Fmoc-protected amino acid) dissolved in DMSO is activated by HATU. After adding base NMM to the mixture, the solution is spotted onto the surface (G5). When different spotting methods were used, droplets of different sizes can be deposited onto the surface, ranging from 50 µm (40 pL, FIG. 8, using piezo inkjet printing) to 1 mm (200 nL, FIG. 9, with contact printing). When Fmoc-protected amino acids are used, the protection group Fmoc can be removed by immersing the array chip into a solution of piperidine in DMF/ethanol. The presence of ethanol enhances the yield of deprotection reaction from < 80 % to close to 100 %. After 6 cycles of washing using DMF/ethanol 1:1 mixture, second building blocks can be coupled to the first building blocks through the same amide formation chemistry. It is important to note that the surface wettability to the polar aprotic solvent remains unchanged after many cycles coupling and deprotection steps (up to 16 cycles).

### Example 5: Matrix Optimization

To demonstrate that the surface G4 is not only compatible with high density spotting technology using different polar aprotic organic solvents, but also applicable for solid phase chemical syntheses, the synthesis of arrays of various small molecular ligands and peptides was investigated. The amount of amino group in coating can be tuned by repeating the C-D cycle (FIG. 2), resulting in 0.072 nmol/cm², 0.364 nmol/cm², and 1.289 nmol nmol/cm² of free amino group for the following synthesis, after 1, 2, and 3 C-D cycles, respectively (G1 in FIGS. 3 and 4). Increasing the chitosan hydrogel coating causes a decrease of surface contact angle for DMSO (from 67° to 46°), however, does not affect the deposition of solvent droplets to the surface.

### Example 6: Linker and Amino Protecting Group Optimization

To investigate the stability of amino protecting group under lipid coupling process and the steric hinderance of the lipophilic groups affecting protein-ligand interaction, we performed six different conditions as shown in FIG. 18. First, we tested four repeat units of β-Ala as linker. The spots without lipophilic group modification showed highest signal, indicating that the linker does not offer enough distance to avoid steric hinderance caused by lipophilic group. With the lipophilic group modification, we observed higher signal when we performed biotin coupling before lipophilic group coupling, indicating that Fmoc as amino protection group is no stable under lipid coupling process. Next, we use six repeat units of β-Ala as linker and Boc as amine protection group. All the spots under three conditions shown similar intensity, indicate that six repeat units of β-Ala as linker and Boc as amino protection group during lipid coupling process are necessary for spot array synthesis.

### Example 7: Protein-ligand Interaction

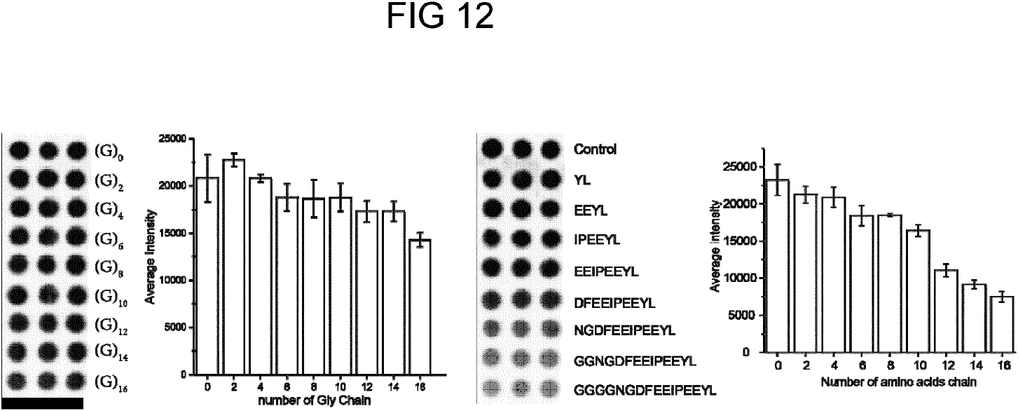

There was synthesized an array of 4 different compounds (biotin, desthiobiotin, iminobiotin and CsA-COOH, a cyclosporin A (CsA) derivative with a carboxylic acid group at position 1). Biotin and desthiobiotin are potent binders to streptavidin, with K_{d} values in the range of pM and low nM, respectively, while iminobiotin is a weak binder to streptavidin with µM dissociation constant. CsA and its derivatives bind to their receptor protein cyclophilin A (CypA) with nM affinity. To increase the coupling yield, each coupling step was repeated two times. Same procedure was used in all amide bond formation reactions. After a capping step using 5% acetic anhydride/2% DIPEA, Fmoc was deprotected by immersing the glass slide in piperidine solution (DMF:ethanol 1:1). All amino groups not in the spotted area were acetylated in the first capping step. Therefore, no amide bond formation would be possible in these area in the following synthesis. The array was then probed by fluorescently labelled streptavidin or CypA (FIGS. 10 and 11). CypA can bind to CsA selectively, but not to biotin and its derivatives. As expected, the fluorescent signal on iminobiotin spot is much weaker than those of biotin and desthiobiotin. At this screening condition, the protein cannot distinguish between pM and low nM binders, while it can distinguish these two strong binders from the weaker µM binder iminobiotin.

### Example 8: Peptide Synthesis

As compared with solid phase peptide synthesis in syringe reactors, array syntheses on planar surface have more exposure to ambient atmosphere. Therefore, the reaction yields are lower than the standard solid phase peptide synthesis [20]. To investigate the efficacy of peptide array synthesis on the G4 surface, there were synthesized peptides with varied length, up to 16 amino acids (FIG. 12) consisting of Gly-residues only (SEQ ID NOs: 1 to 8) or various different amino acids (SEQ ID NOs: 9 to 16). In order to evaluate the coupling yield, biotin was coupled to the N-terminal of every peptide. Thus, only the full length peptides will possess a biotin and be able to bind to fluorescently labelled streptavidin. As shown in FIG. 12, with the increase of peptide lengths, the signals of streptavidin binding decrease gradually. After 16 coupling cycles, the signals for (Gly)₁₆ and thrombin-binding peptide are 69% and 32 % as compared to the spots with biotin directly coupled to the (β-Ala)₆ linker, corresponding to average yields for each coupling step of 98 % and 93%, respectively. Therefore, the surface has shown high compatibility not only for spotting process, but also for solid phase peptide array synthesis.

### Example 9: Peptide-protein Interaction

In order to probe protein-peptide interactions, a peptide was synthesized on the amphiphilic surface. The known calcineurin-binding peptide PVIVIT (SEQ ID NO: 13) and CsA1-COOH were synthesized on the surface. The glass slide was then incubated with fluorescently labelled calcineurin. As shown in FIG. 13, calcineurin can bind specifically to the PVIVIT (SEQ ID NO: 13) but no CsA1-COOH. Therefore, the on-demand peptide array synthesis technology can be used to investigate specific protein-peptide interaction.

### Example 10: Peptide Epitope

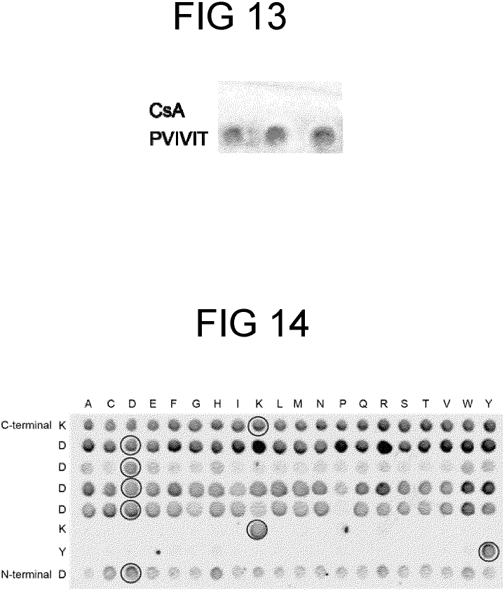

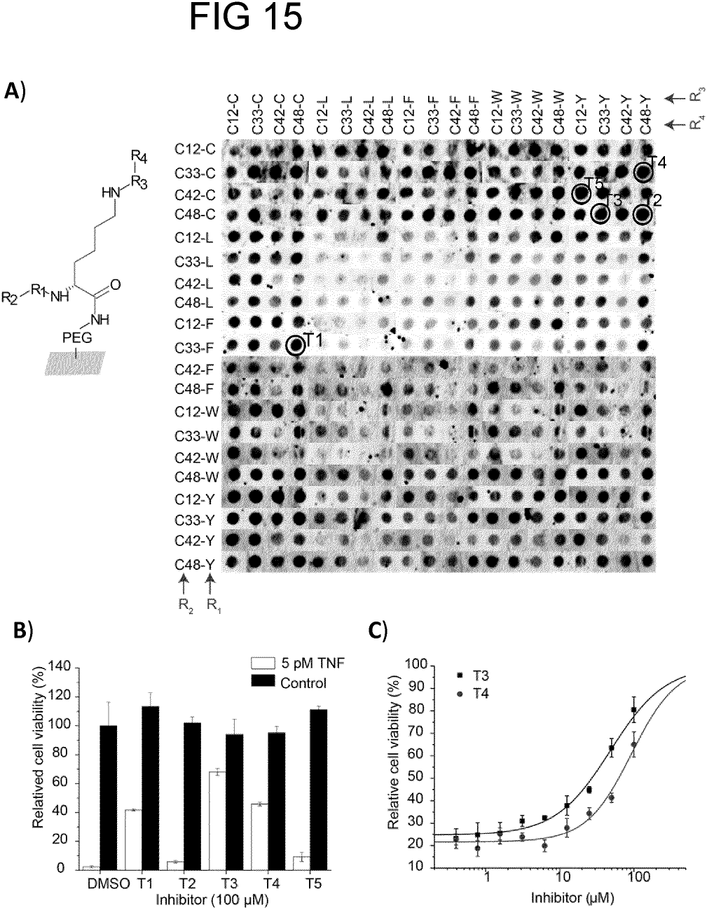

There were also synthesized peptides of SEQ ID NOs: 18-178 on the switchable surface to probe antibody-peptide interaction. Flag-tag peptide and anti-Flag-tag antibody were used as a model system. The flag-tag peptide and its mutations were synthesized on the G4 surface with an (O2Oc)₂-(β-Ala)₄ linker. The glass slide was then incubated with fluorescently labelled anti-flag-tag antibody. As shown in FIG. 14, anti-Flag-tag antibody can bind specifically to the flag-tag peptides, and Y₂ and K₃ are essential for the recognition, in good agreement with the published results [21]. Therefore, the on-demand peptide array synthesis technology can be used to investigate epitope recognition of antibody.

### Example 11: Screening Using Small Molecular Array

There was synthesized a small molecular chemical library of 400 compounds containing up to 4 building blocks and used the array for the discovery of TNF-α inhibitors. TNF-α was labelled with Cy5-NHS and incubated with the small molecular array. Five compounds showing strong fluorescent signal were selected for the following biochemical and cellular analysis, and three compounds can inhibit the cytotoxic effect of TNF-α to L929 cells at 100 µM ( FIGS. 15A, B). As shown in FIG. 15C, two best compounds were select, and they can inhibit the cytotoxic effect of TNF-α to L929 cells in a concentration dependent manner, with an MC₅₀ value of 48 µM and 92 µM, respectively.

### Example 12: Cell Adhesive Biomatrix

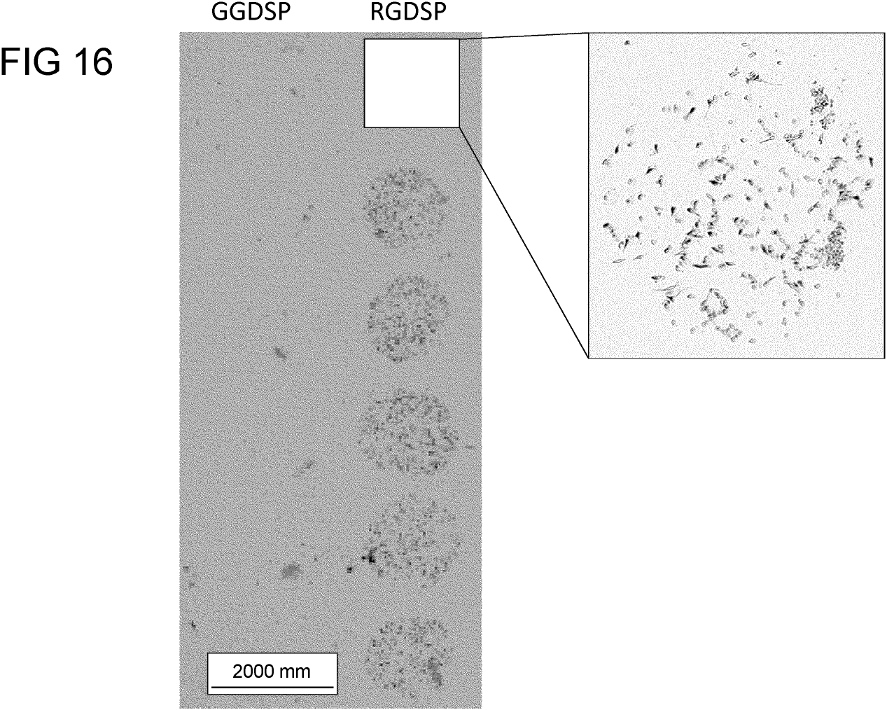

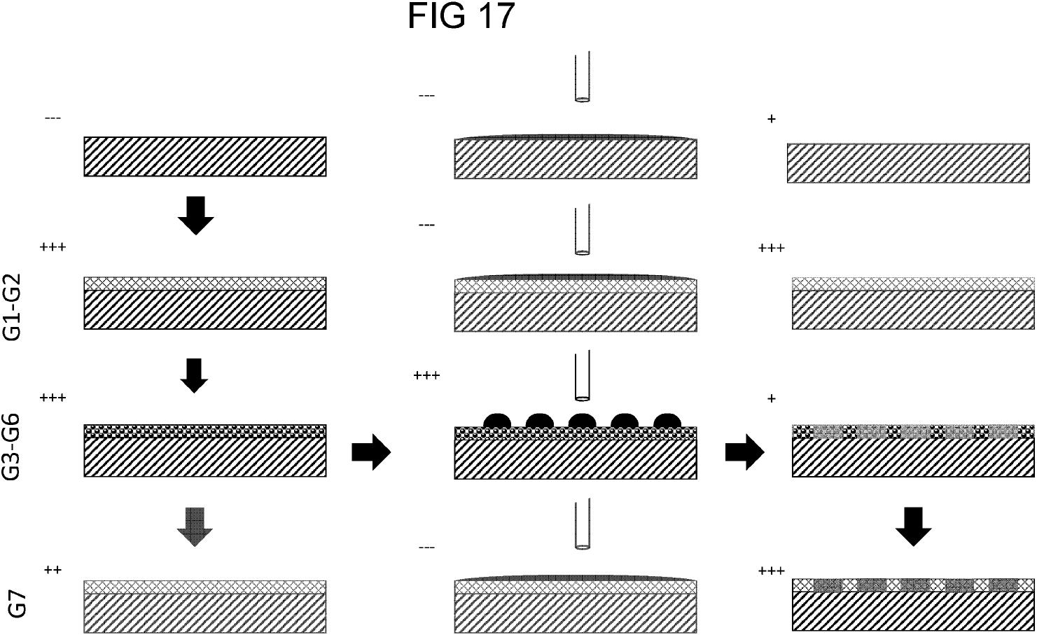

There were synthesized cell adhesive peptide RGDSP (SEQ ID NO: 180) and its mutant GGDSP (SEQ ID NO: 179) as array and investigated their effects on the adhesion to L929 cells. As the chitosan hydrogel coating provides a favorable surface for the adhesion of many types of cells, including L929 cells, the array has been synthesized without removing the lipophilic group. Because of the presence of lipophilic group, the area without peptide as well as the spots with the GGDSP peptide (SEQ ID NO: 179) showed poor adhesion to cells. In contract, the spots with the RGDSP peptide (SEQ ID NO: 180) exhibited strong adhesion to the cells. (FIG. 16) Therefore, the array technology can be used not only for protein-based high throughput screening, but also for developing novel biomaterials as well as cellular assays.

### Discussions

Although different in situ array synthesis methods have their advantages, it is often their disadvantages preventing them from wide application in high throughput screening. Array synthesis on cellulose membrane offers the highest flexibility, however, it is difficult to produce small feature size, preventing the generation of high-density array. Moreover, because of the avoidance of high protein consumption and light scattering caused by cellulose matrices, glass represents a surface superior to cellulose membrane for high throughput screening. While it is difficult to generate small droplets on glass when classical aprotic polar organic solvents such as DMF and DMSO are used, multiple droplets on glass surface also have the tendency to interact with each other and causing droplets movements. Thus, polymer particles have been developed to replace the organic solvents as reaction media. However, the preparation of building block precursors as polymer particles is cumbersome, which also makes the technology expensive and less flexible, limited to a small number of commonly used building blocks such as natural amino acids. An ideal surface for in situ on-demand array synthesis shall fulfill the following requirements: 1) For generating small droplets to create high density array, the surface shall have relatively low wettability to aprotic polar organic solvents; 2) The surface shall have some binding energy to the droplets, to prevent droplet movement such as vapour-mediated droplet motion; 3) the final surface shall be hydrophilic and compatible with various biochemical and cellular assays.

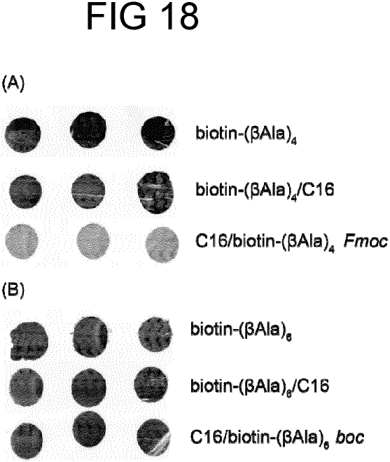

The properties of various surfaces generated in this study and their utilities in in situ array synthesis and the following screening experiments are summarized in FIG. 17. Coating the glass surface with chitosan hydrogel can increase the ligand concentration (G1 and G2), while the hydrophilic coating also provides an environment suitable for developing biochemical and cellular assays. However, the coating does not affect the surface wettability to polar aprotic solvents. When a lipid chain is presented on the surface (G3-G6), it can reduce the wettability remarkably, not only to water, but also to polar aprotic solvents most commonly used in peptide synthesis and combinatorial chemistry (e.g. DMF, DMSO and sulfolane). Droplets of small feature size can be deposited on the surface without spreading. Moreover, in spite of the reduced wettability and increased contact angle, the droplets still possess some binding energy to the amphiphilic surface. Therefore, droplet motion driven by multi-droplet interactions does not occur, even when the droplets are very close to each other with a distance of 40 µm (FIG. 7A). Moreover, the droplets do not move even when the slide was tilted to 90° (FIG. 6). Given that the lipophilic groups will cause remarkable non-specific protein absorption, to switch the surface back to a hydrophilic state would be necessary for some biological applications, especially for detecting specific protein-ligand interactions. The hydrophilic matrices after hydrolyzing the lipid ester bond exhibit high wettability not only to the organic solvents but also to water (G7).

In summary, there was developed a surface coating on glass, compatible with printing of high-density array and classical solid phase combinatorial synthesis. After the synthesis, the relatively hydrophobic surface can be switched to a hydrophilic coating, to make it ideal for protein binding and cell adhesion assays. Other types of chemistry, beyond peptide bond formation is also suitable for synthesizing the array. Examples are Baylis-Hillman reaction[22], Diels-Alderreaction[23], 1,3-Dipolar cycloaddition [24], Henry reaction [25], olefin metathesis [26], multiple component reactions including Ugi reaction and Passerini reaction [27], Nitroaldol reaction[28], Nozaki-Hiyama coupling [29], Paal-Knorr Pyrrole synthesis [30], Prins reaction [31], Sonogashira coupling [32], Staudinger synthesis [33], Stetter reaction [34], as well as various nucleophilic additions and nucleophilic substitutions.

### References

1. Howbrook, D.N., et al., Developments in microarray technologies. Drug Discovery Today, 2003. 8(14): p. 642-651.

2. Joos, T. and P. Kroeger, New frontiers in microarray technology development. Current Opinion in Biotechnology, 2008. 19(1): p. 1-3.

3. Heller, M.J., DNA microarray technology: Devices, systems, and applications. Annual Review of Biomedical Engineering, 2002. 4: p. 129-153.

4. Mayr, L.M. and D. Bojanic, Novel trends in high-throughput screening. Current Opinion in Pharmacology, 2009. 9(5): p. 580-588.

5. Deiss, F., et al., Flow-through synthesis on teflon-patterned paper to produce Peptide arrays for cell-based assays. Angew Chem Int Ed Engl, 2014. 53(25): p. 6374-7.

6. Klim, J.R., et al., A defined glycosaminoglycan-binding substratum for human pluripotent stem cells. Nature Methods, 2010. 7(12): p. 989-U72.

7. Derda, R., et al., Paper-supported 3D cell culture for tissue-based bioassays. Proceedings of the National Academy of Sciences of the United States of America, 2009. 106(44): p. 18457-18462.

8. Lam, K.S., M. Lebl, and V. Krchnak, The “One-Bead-One-Compound” Combinatorial Library Method. Chem Rev, 1997. 97(2): p. 411-448.

9. Kleiner, R.E., C.E. Dumelin, and D.R. Liu, Small-molecule discovery from DNA-encoded chemical libraries. Chemical Society Reviews, 2011.

10. Franzini, R.M., D. Neri, and J. Scheuermann, DNA-Encoded Chemical Libraries: Advancing beyond Conventional Small-Molecule Libraries. Accounts of Chemical Research, 2014. 47(4): p. 1247-1255.

11. Frank, R., Spot-Synthesis - an Easy Technique for the Positionally Addressable, Parallel Chemical Synthesis on a Membrane Support. Tetrahedron, 1992. 48(42): p. 9217-9232.

12. Frank, R. and S. Güler, Verfahren zur schnellen Synthese von trägergebundenen oder freien Peptiden oder Oligonucleotiden, damit hergestelltes Flachmaterial, dessen Verwendung sowie Vorrichtung zur Durchführung des Verfahrens. P, 1990. 40(27): p. 657.9.

13. Fodor, S.P.A., Light-Directed, Spatially Addressable Parallel Chemical Synthesis. Abstracts of Papers of the American Chemical Society, 1991. 202: p. 90-Medi.

14. Pellois, J.P., et al., Individually addressable parallel peptide synthesis on microchips. Nature Biotechnology, 2002. 20(9): p. 922-926.

15. Beyer, M., et al., Combinatorial synthesis of peptide arrays onto a microchip. Science, 2007. 318(5858): p. 1888-1888.

16. Maerkle, F., et al., High-density peptide arrays with combinatorial laser fusing. Adv Mater, 2014. 26(22): p. 3730-4.

17. Stadler, V., et al., Combinatorial synthesis of peptide arrays with a laser printer. Angewandte Chemie-International Edition, 2008. 47(37): p. 7132-7135.

18. Loeffler, F.F., et al., High-flexibility combinatorial peptide synthesis with laser-based transfer of monomers in solid matrix material. Nature Communications, 2016. 7.

19. Cira, N.J., A. Benusiglio, and M. Prakash, Vapour-mediated sensing and motility in two-component droplets. Nature, 2015. 519(7544): p. 446-+.

20. Kramer, A., et al., Spot synthesis: observations and optimizations. Journal of Peptide Research, 1999. 54(4): p. 319-327.

21. Slootstra, J.W., et al., Identification of new tag sequences with differential and selective recognition properties for the anti-FLAG monoclonal antibodies M1, M2 and M5. Molecular Diversity, 1997. 2(3): p. 156-164.

22. Basavaiah, D. and G. Veeraraghavaiah, The Baylis Hillman reaction: a novel concept for creativity in chemistry. Chemical Society Reviews, 2012. 41(1): p. 68-78.

23. Fringuelli, F. and A. Taticchi, The Diels-Alder reaction: selected practical methods. 2002: John Wiley & Sons.

24. Gothelf, K.V. and K.A. Jørgensen, Asymmetric 1, 3-dipolar cycloaddition reactions. Chemical Reviews, 1998. 98(2): p. 863-910.

25. Alvarez-Casao, Y., E. Marques-Lopez, and R.P. Herrera, Organocatalytic Enantioselective Henry Reactions. Symmetry-Basel, 2011. 3(2): p. 220-245.

26. Vougioukalakis, G.C. and R.H. Grubbs, Ruthenium-Based Heterocyclic Carbene-Coordinated Olefin Metathesis Catalysts. Chemical Reviews, 2010. 110(3): p. 1746-1787.

27. Armstrong, R.W., et al., Multiple-component condensation strategies for combinatorial library synthesis. Accounts of Chemical Research, 1996. 29(3): p. 123-131.

28. Meshram, H.M., et al., DMF mediated Henry reaction of isatins: an efficient synthesis of 3-hydroxy-2-oxindole. Green Chemistry Letters and Reviews, 2013. 6(1): p. 19-43.

29. Gil, A., F. Albericio, and M. Alvarez, Role of the Nozaki-Hiyama-Takai-Kishi Reaction in the Synthesis of Natural Products. Chem Rev, 2017. 117(12): p. 8420-8446.

30. Abbat, S., et al., Mechanism of the Paal-Knorr reaction: the importance of water mediated hemialcohol pathway. Rsc Advances, 2015. 5(107): p. 88353-88366.

31. Reddy, B.V.S., et al., The Aza-Prins Reaction in the Synthesis of Natural Products and Analogues. European Journal of Organic Chemistry, 2017. 2017(14): p. 1805-1819.

32. Eckhardt, M. and G.C. Fu, The first applications of carbene ligands in cross-couplings of alkyl electrophiles: Sonogashira reactions of unactivated alkyl bromides and iodides. Journal of the American Chemical Society, 2003. 125(45): p. 13642-13643.

33. Palomo, C., et al., Asymmetric synthesis of beta-lactams through the Staudinger reaction and their use as building blocks of natural and nonnatural products. Current Medicinal Chemistry, 2004. 11(14): p. 1837-1872.

34. de Alaniz, J.R. and T. Rovis, The Catalytic Asymmetric Intramolecular Stetter Reaction. Synlett, 2009(8): p. 1189-1207.

1. An amphiphilic coating suitable for direct and rapid synthesis of an array of peptides and small molecular compounds on a planar surface of a solid support, said amphiphilic coating comprising at least one hydrophilic chemical structure selected from the group consisting of an aminopolysaccharide and an amino acid, and at least one lipophilic group,
said amphiphilic coating exhibiting a wettability contact angle of greater than 20 degrees to a polar aprotic solvent used in the array synthesis and a relatively lower wettability contact angle when said at least one lipophilic group is hydrolyzed.

2. The amphiphilic coating of claim 1, wherein the hydrophilic chemical structure further includes at least one hydroxyl group.

3. The amphiphilic coating of claim 1, wherein the hydrophilic chemical structure is an aminopolysaccharide.

4. The amphiphilic coating of claim 1, wherein the lipophilic group has been conjugated by coupling a fatty acid molecule to a hydroxyl group.

5. The amphiphilic coating of claim 4, wherein the lipophilic group has an acid-labile ether linkage.

6. The amphiphilic coating of claim 1, wherein the lipophilic group comprises an alkyl chain of 4-20 carbon atoms.

7. The amphiphilic coating of claim 1, wherein said lipophilic group is hydrolysable with a base.

8. The amphiphilic coating of claim 1, wherein said lipophilic group is hydrolysable with an acid.

9. A non-porous solid support comprising a planar surface coated with an amphiphilic coating of claim 1.

10. The solid support of claim 9, wherein the amphiphilic coating comprises at least one hydrophilic chemical structure and at least one lipophilic group and wherein the hydrophilic chemical structure further includes at least one hydroxyl group, the solid support includes a linker between the amphiphilic coating on the surface of the solid support and an amino group which is available for subsequent array synthesis.

11. The solid support of claim 10, wherein said linker is a poly-amino acid linker, preferably of the formula (aa)n, wherein aa is an amino acid or a protected amino acid and n is an integer in the range of 3 to 10.

12. The solid support of claim 11, wherein the amino acid aa is selected from the group consisting of glycine, beta-alanine, lysine, serine, threonine, aspartic acid and glutamic acid and mixtures thereof, and wherein said amino acids lysine, serine, threonine, aspartic acid, and glutamic acid are side chain protected.

13. (canceled)

14. The solid support of claim 12, wherein said side chain protected amino acid aa in the linker is protected with a protection group selected from tBu (tert-Butyl) protected serine, tBu protected threonine, tBu protected aspartic acid, tBu protected glutamic acid, and Boe protected lysine.

15. The solid support according to claim 9, wherein the solid support is glass.

16. The solid support according to claim 9, wherein the solid support has a surface-specific loading with amino groups of 1 pmol to 100 nmol per cm².

17. A method for the direct and rapid synthesis of an array of peptides and small molecular compounds on a planar surface of a solid support, wherein said planar surface of a solid support comprises an amphiphilic coating according to claim 1, characterized in that said method comprises the steps:
a) covalently bonding to the amino groups of the amphiphilic coating in predetermined discrete spotting zones, the starting building blocks of the peptides and small molecular compounds to be synthesized, by spotting droplets of a solution comprising chemical reagents reactive to the amino groups onto the predetermined discrete spotting zones, and
b) synthesizing chemical compounds by reacting the first building block with further reactants, in a predetermined sequence and at the predetermined discrete spotting zone, by spotting droplets of a solution comprising chemical reagents reactive to the first building block;
c) obtaining a unitary, single solid support compnsmg an array of different, combinatorically synthesized, bound peptides and small molecular compounds, wherein the chemical structure of said peptides and/or small molecular compounds in said array is different from spot to spot.

18. (canceled)

19. The method of claim 17, wherein the polar aprotic solvent is selected from NMP, DMF, DMSO and sulfolane.

20. The method according to claim 17, wherein a droplet that covers a predetermined discrete spotting zone, has a diameter of 1 µm to 2 mm.

21. The method according to claim 17, wherein blocking and deprotection steps are carried out during synthesis.

22. The method according to claim 17, further comprising as step d) increasing wettability of the amphiphilic coating by hydrolysis of the lipophilic group.

23. (canceled)


SEQUENCE LISTING (180 sequences)

SEQ ID NO: 1 - protein, 2 aa, Artificial sequence
  Synthetic peptide
GG

SEQ ID NO: 2 - protein, 4 aa, Artificial Sequence
  Synthetic peptide
GGGG

SEQ ID NO: 3 - protein, 6 aa, Artificial Sequence
  Synthetic peptide
GGGGGG

SEQ ID NO: 4 - protein, 8 aa, Artificial Sequence
  Synthetic peptide
GGGGGGGG

SEQ ID NO: 5 - protein, 10 aa, Artificial Sequence
  Synthetic peptide
GGGGGGGGGG

SEQ ID NO: 6 - protein, 12 aa, Artificial Sequence
  Synthetic peptide
GGGGGGGGGGGG

SEQ ID NO: 7 - protein, 14 aa, Artificial Sequence
  Synthetic peptide
GGGGGGGGGGGGGG

SEQ ID NO: 8 - protein, 16 aa, Artificial Sequence
  Synthetic peptide
GGGGGGGGGGGGGGGG

SEQ ID NO: 9 - protein, 2 aa, Artificial Sequence
  Synthetic peptide
YL

SEQ ID NO: 10 - protein, 4 aa, Artificial Sequence
  Synthetic peptide
EEYL

SEQ ID NO: 11 - protein, 6 aa, Artificial Sequence
  Synthetic peptide
IPEEYL

SEQ ID NO: 12 - protein, 8 aa, Artificial Sequence
  Synthetic peptide
EEIPEEYL

SEQ ID NO: 13 - protein, 10 aa, Artificial Sequence
  Synthetic peptide
DFEEIPEEYL

SEQ ID NO: 14 - protein, 12 aa, Artificial Sequence
  Synthetic peptide
NGDFEEIPEEYL

SEQ ID NO: 15 - protein, 14 aa, Artificial Sequence
  Synthetic peptide
GGNGDFEEIPEEYL

SEQ ID NO: 16 - protein, 16 aa, Artificial Sequence
  Synthetic peptide
GGGGNGDFEEIPEEYL

SEQ ID NO: 17 - protein, 5 aa, Artificial Sequence
  Synthetic peptide
PIVIT

SEQ ID NO: 18 - protein, 8 aa, Artificial Sequence
  Synthetic peptide
DYKDDDDK

SEQ ID NO: 19 - protein, 8 aa, Artificial Sequence
  Synthetic peptide
AYKDDDDK

SEQ ID NO: 20 - protein, 8 aa, Artificial Sequence
  Synthetic peptide
DAKDDDDK

SEQ ID NO: 21 - protein, 8 aa, Artificial Sequence
  Synthetic peptide
DYADDDDK

SEQ ID NO: 22 - protein, 8 aa, Artificial Sequence
  Synthetic peptide
DYKADDDK

SEQ ID NO: 23 - protein, 8 aa, Artificial Sequence
  Synthetic peptide
DYKDADDK

SEQ ID NO: 24 - protein, 8 aa, Artificial Sequence
  Synthetic peptide
DYKDDADK

SEQ ID NO: 25 - protein, 8 aa, Artificial Sequence
  Synthetic peptide
DYKDDDAK

SEQ ID NO: 26 - protein, 8 aa, Artificial Sequence
  Synthetic peptide
DYKDDDDA

SEQ ID NO: 27 - protein, 8 aa, Artificial Sequence
  Synthetic peptide
CYKDDDDK

SEQ ID NO: 28 - protein, 8 aa, Artificial Sequence
  Synthetic peptide
DCKDDDDK

SEQ ID NO: 29 - protein, 8 aa, Artificial Sequence
  Synthetic peptide
DYCDDDDK

SEQ ID NO: 30 - protein, 8 aa, Artificial Sequence
  Synthetic peptide
DYKCDDDK

SEQ ID NO: 31 - protein, 8 aa, Artificial Sequence
  Synthetic peptide
DYKDCDDK

SEQ ID NO: 32 - protein, 8 aa, Artificial Sequence
  Synthetic peptide
DYKDDCDK

SEQ ID NO: 33 - protein, 8 aa, Artificial Sequence
  Synthetic peptide
DYKDDDCK

SEQ ID NO: 34 - protein, 8 aa, Artificial Sequence
  Synthetic peptide
DYKDDDDC

SEQ ID NO: 35 - protein, 8 aa, Artificial Sequence
  Synthetic peptide
DYKDDDDK

SEQ ID NO: 36 - protein, 8 aa, Artificial Sequence
  Synthetic peptide
DDKDDDDK

SEQ ID NO: 37 - protein, 8 aa, Artificial Sequence
  Synthetic peptide
DYDDDDDK

SEQ ID NO: 38 - protein, 8 aa, Artificial Sequence
  Synthetic peptide
DYKDDDDK

SEQ ID NO: 39 - protein, 8 aa, Artificial Sequence
  Synthetic peptide
DYKDDDDK

SEQ ID NO: 40 - protein, 8 aa, Artificial Sequence
  Synthetic peptide
DYKDDDDK

SEQ ID NO: 41 - protein, 8 aa, Artificial Sequence
  Synthetic peptide
DYKDDDDK

SEQ ID NO: 42 - protein, 8 aa, Artificial Sequence
  Synthetic peptide
DYKDDDDD

SEQ ID NO: 43 - protein, 8 aa, Artificial Sequence
  Synthetic peptide
EYKDDDDK

SEQ ID NO: 44 - protein, 8 aa, Artificial Sequence
  Synthetic peptide
DEKDDDDK

SEQ ID NO: 45 - protein, 8 aa, Artificial Sequence
  Synthetic peptide
DYEDDDDK

SEQ ID NO: 46 - protein, 8 aa, Artificial Sequence
  Synthetic peptide
DYKEDDDK

SEQ ID NO: 47 - protein, 8 aa, Artificial Sequence
  Synthetic peptide
DYKDEDDK

SEQ ID NO: 48 - protein, 8 aa, Artificial Sequence
  Synthetic peptide
DYKDDEDK

SEQ ID NO: 49 - protein, 8 aa, Artificial Sequence
  Synthetic peptide
DYKDDDEK

SEQ ID NO: 50 - protein, 8 aa, Artificial Sequence
  Synthetic peptide
DYKDDDDE

SEQ ID NO: 51 - protein, 8 aa, Artificial Sequence
  Synthetic peptide
FYKDDDDK

SEQ ID NO: 52 - protein, 8 aa, Artificial Sequence
  Synthetic peptide
DFKDDDDK

SEQ ID NO: 53 - protein, 8 aa, Artificial Sequence
  Synthetic peptide
DYFDDDDK

SEQ ID NO: 54 - protein, 8 aa, Artificial Sequence
  Synthetic peptide
DYKFDDDK

SEQ ID NO: 55 - protein, 8 aa, Artificial Sequence
  Synthetic peptide
DYKDFDDK

SEQ ID NO: 56 - protein, 8 aa, Artificial Sequence
  Synthetic peptide
DYKDDFDK

SEQ ID NO: 57 - protein, 8 aa, Artificial Sequence
  Synthetic peptide
DYKDDDFK

SEQ ID NO: 58 - protein, 8 aa, Artificial Sequence
  Synthetic peptide
DYKDDDDF

SEQ ID NO: 59 - protein, 8 aa, Artificial Sequence
  Synthetic peptide
GYKDDDDK

SEQ ID NO: 60 - protein, 8 aa, Artificial Sequence
  Synthetic peptide
DGKDDDDK

SEQ ID NO: 61 - protein, 8 aa, Artificial Sequence
  Synthetic peptide
DYGDDDDK

SEQ ID NO: 62 - protein, 8 aa, Artificial Sequence
  Synthetic peptide
DYKGDDDK

SEQ ID NO: 63 - protein, 8 aa, Artificial Sequence
  Synthetic peptide
DYKDGDDK

SEQ ID NO: 64 - protein, 8 aa, Artificial Sequence
  Synthetic peptide
DYKDDGDK

SEQ ID NO: 65 - protein, 8 aa, Artificial Sequence
  Synthetic peptide
DYKDDDGK

SEQ ID NO: 66 - protein, 8 aa, Artificial Sequence
  Synthetic peptide
DYKDDDDG

SEQ ID NO: 67 - protein, 8 aa, Artificial Sequence
  Synthetic peptide
HYKDDDDK

SEQ ID NO: 68 - protein, 8 aa, Artificial Sequence
  Synthetic peptide
DHKDDDDK

SEQ ID NO: 69 - protein, 8 aa, Artificial Sequence
  Synthetic peptide
DYHDDDDK

SEQ ID NO: 70 - protein, 8 aa, Artificial Sequence
  Synthetic peptide
DYKHDDDK

SEQ ID NO: 71 - protein, 8 aa, Artificial Sequence
  Synthetic peptide
DYKDHDDK

SEQ ID NO: 72 - protein, 8 aa, Artificial Sequence
  Synthetic peptide
DYKDDHDK

SEQ ID NO: 73 - protein, 8 aa, Artificial Sequence
  Synthetic peptide
DYKDDDHK

SEQ ID NO: 74 - protein, 8 aa, Artificial Sequence
  Synthetic peptide
DYKDDDDH

SEQ ID NO: 75 - protein, 8 aa, Artificial Sequence
  Synthetic peptide
IYKDDDDK

SEQ ID NO: 76 - protein, 8 aa, Artificial Sequence
  Synthetic peptide
DIKDDDDK

SEQ ID NO: 77 - protein, 8 aa, Artificial Sequence
  Synthetic peptide
DYIDDDDK

SEQ ID NO: 78 - protein, 8 aa, Artificial Sequence
  Synthetic peptide
DYKIDDDK

SEQ ID NO: 79 - protein, 8 aa, Artificial Sequence
  Synthetic peptide
DYKDIDDK

SEQ ID NO: 80 - protein, 8 aa, Artificial Sequence
  Synthetic peptide
DYKDDIDK

SEQ ID NO: 81 - protein, 8 aa, Artificial Sequence
  Synthetic peptide
DYKDDDIK

SEQ ID NO: 82 - protein, 8 aa, Artificial Sequence
  Synthetic peptide
DYKDDDDI

SEQ ID NO: 83 - protein, 8 aa, Artificial Sequence
  Synthetic peptide
KYKDDDDK

SEQ ID NO: 84 - protein, 8 aa, Artificial Sequence
  Synthetic peptide
DKKDDDDK

SEQ ID NO: 85 - protein, 8 aa, Artificial Sequence
  Synthetic peptide
DYKDDDDK

SEQ ID NO: 86 - protein, 8 aa, Artificial Sequence
  Synthetic peptide
DYKKDDDK

SEQ ID NO: 87 - protein, 8 aa, Artificial Sequence
  Synthetic peptide
DYKDKDDK

SEQ ID NO: 88 - protein, 8 aa, Artificial Sequence
  Synthetic peptide
DYKDDKDK

SEQ ID NO: 89 - protein, 8 aa, Artificial Sequence
  Synthetic peptide
DYKDDDKK

SEQ ID NO: 90 - protein, 8 aa, Artificial Sequence
  Synthetic peptide
DYKDDDDK

SEQ ID NO: 91 - protein, 8 aa, Artificial Sequence
  Synthetic peptide
LYKDDDDK

SEQ ID NO: 92 - protein, 8 aa, Artificial Sequence
  Synthetic peptide
DLKDDDDK

SEQ ID NO: 93 - protein, 8 aa, Artificial Sequence
  Synthetic peptide
DYLDDDDK

SEQ ID NO: 94 - protein, 8 aa, Artificial Sequence
  Synthetic peptide
DYKLDDDK

SEQ ID NO: 95 - protein, 8 aa, Artificial Sequence
  Synthetic peptide
DYKDLDDK

SEQ ID NO: 96 - protein, 8 aa, Artificial Sequence
  Synthetic peptide
DYKDDLDK

SEQ ID NO: 97 - protein, 8 aa, Artificial Sequence
  Synthetic peptide
DYKDDDLK

SEQ ID NO: 98 - protein, 8 aa, Artificial Sequence
  Synthetic peptide
DYKDDDDL

SEQ ID NO: 99 - protein, 8 aa, Artificial Sequence
  Synthetic peptide
MYKDDDDK

SEQ ID NO: 100 - protein, 8 aa, Artificial Sequence
  Synthetic peptide
DMKDDDDK

SEQ ID NO: 101 - protein, 8 aa, Artificial Sequence
  Synthetic peptide
DYMDDDDK

SEQ ID NO: 102 - protein, 8 aa, Artificial Sequence
  Synthetic peptide
DYKMDDDK

SEQ ID NO: 103 - protein, 8 aa, Artificial Sequence
  Synthetic peptide
DYKDMDDK

SEQ ID NO: 104 - protein, 8 aa, Artificial Sequence
  Synthetic peptide
DYKDDMDK

SEQ ID NO: 105 - protein, 8 aa, Artificial Sequence
  Synthetic peptide
DYKDDDMK

SEQ ID NO: 106 - protein, 8 aa, Artificial Sequence
  Synthetic peptide
DYKDDDDM

SEQ ID NO: 107 - protein, 8 aa, Artificial Sequence
  Synthetic peptide
NYKDDDDK

SEQ ID NO: 108 - protein, 8 aa, Artificial Sequence
  Synthetic peptide
DNKDDDDK

SEQ ID NO: 109 - protein, 8 aa, Artificial Sequence
  Synthetic peptide
DYNDDDDK

SEQ ID NO: 110 - protein, 8 aa, Artificial Sequence
  Synthetic peptide
DYKNDDDK

SEQ ID NO: 111 - protein, 8 aa, Artificial Sequence
  Synthetic peptide
DYKDNDDK

SEQ ID NO: 112 - protein, 8 aa, Artificial Sequence
  Synthetic peptide
DYKDDNDK

SEQ ID NO: 113 - protein, 8 aa, Artificial Sequence
  Synthetic peptide
DYKDDDNK

SEQ ID NO: 114 - protein, 8 aa, Artificial Sequence
  Synthetic peptide
DYKDDDDN

SEQ ID NO: 115 - protein, 8 aa, Artificial Sequence
  Synthetic peptide
PYKDDDDK

SEQ ID NO: 116 - protein, 8 aa, Artificial Sequence
  Synthetic peptide
DPKDDDDK

SEQ ID NO: 117 - protein, 8 aa, Artificial Sequence
  Synthetic peptide
DYPDDDDK

SEQ ID NO: 118 - protein, 8 aa, Artificial Sequence
  Synthetic peptide
DYKPDDDK

SEQ ID NO: 119 - protein, 8 aa, Artificial Sequence
  Synthetic peptide
DYKDPDDK

SEQ ID NO: 120 - protein, 8 aa, Artificial Sequence
  Synthetic peptide
DYKDDPDK

SEQ ID NO: 121 - protein, 8 aa, Artificial Sequence
  Synthetic peptide
DYKDDDPK

SEQ ID NO: 122 - protein, 8 aa, Artificial Sequence
  Synthetic peptide
DYKDDDDP

SEQ ID NO: 123 - protein, 8 aa, Artificial Sequence
  Synthetic peptide
QYKDDDDK

SEQ ID NO: 124 - protein, 8 aa, Artificial Sequence
  Synthetic peptide
DQKDDDDK

SEQ ID NO: 125 - protein, 8 aa, Artificial Sequence
  Synthetic peptide
DYQDDDDK

SEQ ID NO: 126 - protein, 8 aa, Artificial Sequence
  Synthetic peptide
DYKQDDDK

SEQ ID NO: 127 - protein, 8 aa, Artificial Sequence
  Synthetic peptide
DYKDQDDK

SEQ ID NO: 128 - protein, 7 aa, Artificial Sequence
  Synthetic peptide
DYKDDQK

SEQ ID NO: 129 - protein, 8 aa, Artificial Sequence
  Synthetic peptide
DYKDDDQK

SEQ ID NO: 130 - protein, 8 aa, Artificial Sequence
  Synthetic peptide
DYKDDDDQ

SEQ ID NO: 131 - protein, 8 aa, Artificial Sequence
  Synthetic peptide
RYKDDDDK

SEQ ID NO: 132 - protein, 8 aa, Artificial Sequence
  Synthetic peptide
DRKDDDDK

SEQ ID NO: 133 - protein, 8 aa, Artificial Sequence
  Synthetic peptide
DYRDDDDK

SEQ ID NO: 134 - protein, 8 aa, Artificial Sequence
  Synthetic peptide
DYKRDDDK

SEQ ID NO: 135 - protein, 8 aa, Artificial Sequence
  Synthetic peptide
DYKDRDDK

SEQ ID NO: 136 - protein, 8 aa, Artificial Sequence
  Synthetic peptide
DYKDDRDK

SEQ ID NO: 137 - protein, 8 aa, Artificial Sequence
  Synthetic peptide
DYKDDDRK

SEQ ID NO: 138 - protein, 8 aa, Artificial Sequence
  Synthetic peptide
DYKDDDDR

SEQ ID NO: 139 - protein, 8 aa, Artificial Sequence
  Synthetic peptide
SYKDDDDK

SEQ ID NO: 140 - protein, 8 aa, Artificial Sequence
  Synthetic peptide
DSKDDDDK

SEQ ID NO: 141 - protein, 8 aa, Artificial Sequence
  Synthetic peptide
DYSDDDDK

SEQ ID NO: 142 - protein, 8 aa, Artificial Sequence
  Synthetic peptide
DYKSDDDK

SEQ ID NO: 143 - protein, 8 aa, Artificial Sequence
  Synthetic peptide
DYKDSDDK

SEQ ID NO: 144 - protein, 8 aa, Artificial Sequence
  Synthetic peptide
DYKDDSDK

SEQ ID NO: 145 - protein, 8 aa, Artificial Sequence
  Synthetic peptide
DYKDDDSK

SEQ ID NO: 146 - protein, 8 aa, Artificial Sequence
  Synthetic peptide
DYKDDDDS

SEQ ID NO: 147 - protein, 8 aa, Artificial Sequence
  Synthetic peptide
TYKDDDDK

SEQ ID NO: 148 - protein, 8 aa, Artificial Sequence
  Synthetic peptide
DTKDDDDK

SEQ ID NO: 149 - protein, 8 aa, Artificial Sequence
  Synthetic peptide
DYTDDDDK

SEQ ID NO: 150 - protein, 8 aa, Artificial Sequence
  Synthetic peptide
DYKTDDDK

SEQ ID NO: 151 - protein, 8 aa, Artificial Sequence
  Synthetic peptide
DYKDTDDK

SEQ ID NO: 152 - protein, 8 aa, Artificial Sequence
  Synthetic peptide
DYKDDTDK

SEQ ID NO: 153 - protein, 8 aa, Artificial Sequence
  Synthetic peptide
DYKDDDDT

SEQ ID NO: 154 - protein, 8 aa, Artificial Sequence
  Synthetic peptide
DYKDDDDT

SEQ ID NO: 155 - protein, 8 aa, Artificial Sequence
  Synthetic peptide
VYKDDDDK

SEQ ID NO: 156 - protein, 8 aa, Artificial Sequence
  Synthetic peptide
DVKDDDDK

SEQ ID NO: 157 - protein, 8 aa, Artificial Sequence
  Synthetic peptide
DYVDDDDK

SEQ ID NO: 158 - protein, 8 aa, Artificial Sequence
  Synthetic peptide
DYKVDDDK

SEQ ID NO: 159 - protein, 8 aa, Artificial Sequence
  Synthetic peptide
DYKDVDDK

SEQ ID NO: 160 - protein, 8 aa, Artificial Sequence
  Synthetic peptide
DYKDDVDK

SEQ ID NO: 161 - protein, 8 aa, Artificial Sequence
  Synthetic peptide
DYKDDDVK

SEQ ID NO: 162 - protein, 8 aa, Artificial Sequence
  Synthetic peptide
DYKDDDDV

SEQ ID NO: 163 - protein, 8 aa, Artificial Sequence
  Synthetic peptide
WYKDDDDK

SEQ ID NO: 164 - protein, 8 aa, Artificial Sequence
  Synthetic peptide
DWKDDDDK

SEQ ID NO: 165 - protein, 8 aa, Artificial Sequence
  Synthetic peptide
DYWDDDDK

SEQ ID NO: 166 - protein, 8 aa, Artificial Sequence
  Synthetic peptide
DYKWDDDK

SEQ ID NO: 167 - protein, 8 aa, Artificial Sequence
  Synthetic peptide
DYKDWDDK

SEQ ID NO: 168 - protein, 8 aa, Artificial Sequence
  Synthetic peptide
DYKDDWDK

SEQ ID NO: 169 - protein, 8 aa, Artificial Sequence
  Synthetic peptide
DYKDDDWK

SEQ ID NO: 170 - protein, 8 aa, Artificial Sequence
  Synthetic peptide
DYKDDDDW

SEQ ID NO: 171 - protein, 8 aa, Artificial Sequence
  Synthetic peptide
YYKDDDDK

SEQ ID NO: 172 - protein, 8 aa, Artificial Sequence
  Synthetic peptide
DYKDDDDK

SEQ ID NO: 173 - protein, 8 aa, Artificial Sequence
  Synthetic peptide
DYYDDDDK

SEQ ID NO: 174 - protein, 8 aa, Artificial Sequence
  Synthetic peptide
DYKYDDDK

SEQ ID NO: 175 - protein, 8 aa, Artificial Sequence
  Synthetic peptide
DYKDYDDK

SEQ ID NO: 176 - protein, 8 aa, Artificial Sequence
  Synthetic peptide
DYKDDYDK

SEQ ID NO: 177 - protein, 8 aa, Artificial Sequence
  Synthetic peptide
DYKDDDYK

SEQ ID NO: 178 - protein, 8 aa, Artificial Sequence
  Synthetic peptide
DYKDDDDY

SEQ ID NO: 179 - protein, 5 aa, Artificial Sequence
  Synthetic peptide
GGDSP

SEQ ID NO: 180 - protein, 5 aa, Artificial Sequence
  Synthetic peptide
RGDSP